(19) United States
(12) Patent Application Publication
(10) Pub. No.: US 2026/0357167 A1
(43) Pub. Date: Nov. 12, 2026

(54) POPULATION OF PARTICLES, METHOD FOR PREPARATION AND USES THERE

(71) Applicant: GIGABLUE LTD, Manof (IL)

(72) Inventors: Levy DOTAN, Manof (IL); Sapir MARKUS, Givat Yearim (IL)

(73) Assignee: GIGABLUE LTD, Manof (IL)

(21) Appl. No.: 19/118,674

(22) PCT Filed: Oct. 5, 2023
(86) PCT No.: PCT/IL2023/051064

§ 371 Date: Apr. 4, 2025

Related U.S. Application Data
(60) Provisional application No. 63/378,524, filed on Oct. 6, 2022.

(51) Int. Cl. C12M 1/00; C12M 1/04; C12M 1/12; C12M 1/34

(52) U.S. Cl. CPC C12M 21/02; C12M 23/02; C12M 23/20; C12M 23/24; C12M 25/16; C12M 41/34

(57) ABSTRACT

The present disclosure provides a population of particles, methods for their preparation and uses thereof, e.g. for carbon dioxide sequestration. Each particle comprises a construct comprising at least one solid core, optionally, at least one barrier coating layer over the at least one solid core, a scaffold associated at least at the outer surface of the at least one solid core or of the barrier coating, if the barrier coating is present in the construct, the scaffold being suitable for support growth of photosynthesizing aquatic organism and gas entrapped within the at least one solid core, the gas having a first specific gravity less than that of water and present in an amount sufficient to provide floatation of the particle, once the particle is brought into contact with the water, the construct having a second specific gravity greater than that of water; and the at least one solid core, or the barrier coating, if present in the construct, having a permeability configured to allow controlled exchange between the entrapped gas and water external to the construct.

### TECHNOLOGICAL FIELD

The present disclosure is in the field of chemistry and carbon dioxide sequestration.

### BACKGROUND ART

References considered to be relevant as background to the presently disclosed subject matter are listed below:
International Patent Application Publication No. WO2021126315
U.S. Pat. No. 8,033,879
US Patent Application Publication No. 20080236033
U.S. Pat. No. 5,965,117
Great Britain Patent Application Publication No. 2337749
U.S. Pat. No. 1,0752,528
European Patent No. EP1207743
Chinese Patent Application No. CN115428726
U.S. Pat. No. 8,753,863

Acknowledgement of the above references herein is not to be inferred as meaning that these are in any way relevant to the patentability of the presently disclosed subject matter.

### BACKGROUND

WO2021126315 discloses a nano bio-composite nutrient carrier containing a water-soluble polymer with an iron nutrient, nourishing an aquatic organism. The water-soluble polymer includes a hydrogen bonded interpenetrating polymer network entrapping the iron nutrient. The nutrient carrier is buoyant having a density of <1.0 grams per cubic centimeter.

U.S. Pat. No. 8,033,879 discloses compositions, methods, and equipment for biological and physical geoengineering. It introduces inorganic particles or floats designed for dispersal on water bodies. These compositions offer benefits such as improving yields in pelagic aquaculture, increasing carbon sequestration, and combating global warming by raising surface albedo and promoting cloud formation.

US20080236033 discloses floating slow-release fertilizer which enables the growth of phytoplankton in ocean thereby removing CO₂ from atmosphere.

U.S. Pat. No. 5,965,117 discloses water-buoyant compositions comprising a source of micronutrients for photosynthetic phytoplankton growth which are useful for stimulating photosynthetic phytoplankton growth in ocean areas devoid of such growth when deployed on ocean surfaces as floating particles.

GB2337749 discloses a method for clearing up algal blooms, comprising the steps of sinking the algal cells, organic pollutants and over-enriched nutrients to the bottom sea using iron oxides enriched particles that are modified by cationic reagents.

U.S. Pat. No. 10,752,528 discloses microorganism-containing biocatalysts which have a large population of the microorganisms irreversibly retained in the interior thereof.

EP1207743 discloses a method of increasing seafood production in the oceans the method comprises testing the water at the surface of the ocean in order to determine the nutrients that are missing, applying to the surface of the ocean a fertilizer that comprises an iron chelate.

CN115428726 discloses a method for sequestering carbon dioxide in the ocean using phosphorus supplementation. The method involves transporting a phosphorus source loader to a nutrient-poor area, releasing it on a continental shelf, and dispersing phosphorus into the ocean. The nutrient mix promotes phytoplankton growth in the euphotic layer, which converts atmospheric carbon dioxide into organic matter thereby reducing atmospheric concentration of carbon dioxide.

U.S. Pat. No. 8,753,863 describes a method for removing carbon dioxide from the atmosphere. The method comprises the step of delivering urea from a floating vessel to a region of a photic zone of the ocean, whereby the number of phytoplankton is caused to increase in the region upon addition of the urea.

### General Description

The present disclosure provides, in accordance with a first of its aspects, a population of particles, each particle comprising a construct comprising:
at least one solid core,
optionally, at least one barrier coating layer over the at least one solid core, and
a scaffold associated at least at the outer surface of said at least one solid core or of said barrier coating, if said barrier coating is present in the construct, the scaffold being suitable for support growth of photosynthesizing aquatic organism;
gas entrapped within said at least one solid core, the gas having a first specific gravity less than that of water and present in an amount sufficient to provide floatation of said particle, once the particle is brought into contact with the water;
said construct having a second specific gravity greater than that of water; and
said at least one solid core, or said barrier coating (if present in said construct), having a permeability configured to allow controlled exchange between the entrapped gas and water external to said construct.

The present disclosure provides, in accordance with a second of its aspects, a method of producing particles, the method comprising mixing solid core material, optionally having at least one layer of barrier coating over the solid core, with a scaffold forming material that is suitable for support growth of photosynthesizing aquatic organism, under conditions suitable to allow association of a scaffold to at least an outer surface of the solid core;
said solid core material comprises gas entrapped therein, the gas having a first specific gravity less than that of water and is in an amount sufficient to result in floatation of said particle, once the particle is brought into contact with the water;
the combination of at least one solid core, said at least one layer of barrier coating, if present, and the scaffold having a second specific gravity greater than that of water; and
the combination of the solid core, the at least one layer of barrier coating, if present, the scaffold and the gas is selected to provide, in the resulting particle, controlled exchange between the entrapped gas and water external to said construct, once said particle is brought into contact with water.

The present disclosure provides, in accordance with a second of its aspects, a method for carbon dioxide sequestration, comprising distribution of particles over a selected area of body of water comprising at least one photosynthesizing aquatic organism and being open to a source of carbon dioxide to be sequestered;
wherein each of said particles comprise a construct comprising:
   at least one solid core,
   optionally, at least one barrier coating layer over the at least one solid core, and
   a scaffold associated at least at the outer surface of said at least one solid core or of said barrier coating, if said barrier coating is present in the construct, the scaffold being suitable for support growth of photosynthesizing aquatic organism;
   gas entrapped within said at least one solid core, the gas having a first specific gravity less than that of water and present in an amount sufficient to provide floatation of said particle, once the particle is brought into contact with the water;
said construct having a second specific gravity greater than that of water; and
said at least one solid core, or said barrier coating, if present in said construct, having a permeability configured to allow controlled exchange between the entrapped gas and water external to said construct.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1I are schematic illustrations of constructs according to some examples of the presently disclosed subject matter.

FIGS. 2A-2B include a top view image (FIG. 2A) and a corresponding binary black and white image (FIG. 2B) of an alginate-coated vermiculite particles as observed in a floating-sinking experiment according to some examples of the present disclosure.

FIG. 3 is a plot showing results of floating-sinking experiments (represented as change of percentage of floating particles over time) for vermiculite particles coated with changing concentrations of calcium alginate according to some examples of the present disclosure. Error bars represent standard deviation.

FIG. 4 is a plot showing results of floating-sinking experiments (represented as change of percentage of floating particles over time) for vermiculite particles coated with different number of layers of calcium alginate according to some examples of the present disclosure. Error bars represent standard deviation.

FIGS. 5A-5C are micrographs showing cross-sectional views (FIG. 5A and FIG. 5B) as well as complete particle representation (FIG. 5C) of floating 2% alginate-coated vermiculite particles stained with fluorescein, according to some examples of the present disclosure.

FIG. 6 is a micrograph showing sunken 4% alginate-coated vermiculite particles, according to some examples of the present disclosure.

FIG. 7 is a bar plot representing microalgae dry biomass as a function of growing conditions on Tuff scaffold (“Tuff”), without Fe+Mn supplementation and with high and low concentration of the macronutrients in medium (“Tuff C HN” or “Tuff C LN”, respectively), or with Fe+Mn supplementation and with high and low concentration of the macronutrients in medium (“Tuff Fe Mn HN” or “Tuff Fe Mn LN”, respectively), plotted using Fluorescence Activated Cell Sorter (FACS) or Hemocytometer.

FIGS. 8A-8B are microphotographs showing fluorescent microalgae attached on tuff particulates according to some examples of present disclosure: FIG. 8A shows control tuff particulates in high nutrients medium, FIG. 8B shows Fe+Mn-supplemented tuff particulates in high nutrients medium.

FIG. 9 is a micrograph showing microalgae attached and entrapped within Fe+Mn-supplemented cotton fibers in high nutrients medium according to some examples of present disclosure.

FIGS. 10A-10D are a micrograph showing vermiculite particles coated with alginate and cannabus fibers (FIG. 10A) according to some examples of present disclosure; a micrograph showing vermiculite particles coated with alginate and cotton fibers (FIG. 10B) according to some examples of present disclosure; a micrograph showing vermiculite particles coated with alginate and tuff dust (FIG. 10C); and a photograph showing vermiculite particles coated with alginate and cotton fibers floating in water (FIG. 10D) according to some examples of present disclosure.

FIG. 11 is a plot showing the results of fiber2particle mathematical model simulation according to a non-limiting example of present disclosure: where the simulated biomass growth (solid line) is shown to be fitted to experimental growth data (dots).

### DETAILED DESCRIPTION

Generally, the present disclosure is based on the development of particles having, inter alia, a control floatation or buoyancy property, resulting in floatation of the particles over water, and a pre-designed and controllable settling or sedimentation property, triggering the particles the settle within the water.

Further, the developed particles are designed to support growth of photosynthesizing aquatic organism which contribute on the one hand to the sequestration of carbon dioxide, and on the other hand, can contribute to the settling of the particles in the water.

Thus, when in proximity with photosynthesizing aquatic organism, e.g. those harboring the sunlight zone (photic zone) of bodies of water, the presently disclosed floating particles can serve as a support scaffold for these algae until actuation of the pre-designed settling trigger, which then results in the process of particles' settling within the body of water, together with the harboring algae.

Thus, in the context of a first aspect of the presently disclosed subject matter, there is provides a population of particles, each particle comprising a construct comprising:
at least one solid core,
optionally, at least one barrier coating layer over the at least one solid core, and
a scaffold associated at least at the outer surface of the at least one solid core or of the barrier coating, if the barrier coating is present in the construct, the scaffold being suitable for supporting growth of photosynthesizing aquatic organism;
gas entrapped within the at least one solid core, the gas having a first specific gravity less than that of water and present in an amount sufficient to provide floatation of the particle, once the particle is brought into contact with the water;
the construct having a second specific gravity greater than that of water; and
the at least one solid core, or the barrier coating, if present in the construct, having a permeability configured to allow controlled exchange between the entrapped gas and water external to the construct.

In the context of the presently disclosed subject matter the term “population of particles” denote two or more particles, preferably multiplicity of particles, which while all have the same construction as defined above, may not be necessarily the same in the population. In other words, while all have a solid core, gas entrapped at least in the core, and scaffold as defined herein, some may or may not have the barrier layer, some may differ in the solid core, some may differ in the type of gas entrapped, some may differ in the type of scaffold, some may differ in the number of barrier layers, some may differ in dimensions, etc.

The particles are a construct of components. Thus, it is appreciated that that term “construct” as used herein, denotes a structured solid object formed by an organized assembly of the indicated components, including at least the solid core, the gas and the scaffold.

Each particle in the population of particles comprise at least one solid core. In the context of the presently disclosed subject matter, the term “solid core” should be understood to encompass any non-flowing substance, including, rocks, minerals, glass, as well as gel like materials, as further discussed below. The solid core is a discrete solid entity within the construct, that is distinguishable (e.g. visually or using imaging techniques) at least from the growth supporting scaffold. In some examples, when the construct comprises also at least one layer of the barrier coating, the solid core is also distinguishable from the barrier layer.

In some examples of the presently disclosed subject matter, the solid core is a water insoluble particulate matter. This means that once brought into contact with water, the solid core per se will not immediately dissolve.

In some examples of the presently disclosed subject matter, the solid core is an expanded particulate and/or a porous particulate.

In the context of the presently disclosed subject matter, the terms “expanded” or “porous”, with reference to the solid core, may be understood to refer to having voids, preferably holding the entrapped gas. Thus, when referring to an expanded and/or porous particulate it is to be understood to encompass any particulate that contains voids/open gas-containing spaces. These voids can be distributed throughout the material, and the size, shape, distribution, and interconnectivity of the voids can vary between the particulates. The voids can have a shape of cavities within the substance, can represent spaces between layers in a layered substance or any other form of voids within the particulate.

In some examples of the presently disclosed subject matter, the term “expanded” denotes a material, e.g. a mineral that has undergone a thermal process resulting in its expansion. Accordingly, and as an example, expanded vermiculite is raw vermiculite that underwent a process involving exposure to high temperatures (also known by the term exfoliation) causing expansion of its inner layers and turning into a form of lightweight, porous material with layered structure.

Thus, in some examples of the presently disclosed subject matter, the solid core is an expanded particulate (e.g. where the raw material underwent the exfoliation process, resulting in a porous layered structure that can hold the said gas).

In the context of the presently disclosed subject matter, when referring to an expanded particulate, it is to be understood to refer to particulate matter that has expanded as a result of treatment, e.g. heat treatment. In some examples, the particulate material is expanded due to a process of exfoliation.

In some examples of the presently disclosed subject matter, the solid core is an expanded particulate mineral.

In the context of the presently disclosed subject matter, the term “mineral” should be understood to have its regular meaning, as known in the art. For example, the term “mineral” can be understood to pertain to inorganic, crystalline substance, including singular crystalline entities and aggregates thereof. In some examples of the presently disclosed subject matter, the term “mineral” includes compounds that constitute geological formations and substrates. It is acknowledged that “minerals” may also arise from the alteration or fusion of their constituent components, resulting in the formation of new chemical entities within the context of rocks.

A non-limiting list of expanded/porous particulate mineral include vermiculite (including specifically expanded vermiculite), montmorillonite, bentonite, hectorite, saponite, kaolinite, halloysite, illite, palygorskite, sepiolite and nontronite.

In some examples of the presently disclosed subject matter, the solid core is expanded vermiculite mineral.

In some examples of the presently disclosed subject matter, the solid core is an expanded particulate volcanic glass.

A non-limiting list of expandable particulate volcanic glass include perlite and pumice.

In some examples of the presently disclosed subject matter, the solid core is expanded particulate perlite.

In some examples of the presently disclosed subject matter, the solid core is expanded particulate pumice.

In some examples of the presently disclosed subject matter, the solid core is a porous organic particulate.

The organic core can be syntenic or non-synthetic.

In some examples of the presently disclosed subject matter, the organic solid core is a carbon-based sponge.

In some examples of the presently disclosed subject matter, the organic solid is a natural/non-synthetic carbon-based sponge.

A non-limiting list of organic (non-synthetic) sponge that can be utilized in the context of the presently disclosed subject matter include sea sponge, cellulose based sponge, loofah sponge and combinations of same.

In some examples of the presently disclosed subject matter, the organic solid core is a carbon-based foam.

A non-limiting list of carbon-based foams that can be utilized in the context of the presently disclosed subject matter, include foamed polyurethane or latex (yet, preferably polyurethane).

In some examples of the presently disclosed subject matter, the organic solid core comprises a carbon-based fibrous (porous) material.

A non-limiting list of carbon-based fibrous material that can be used as the solid core in the context of the presently disclosed subject matter include coir fibers, rise husk, wood fibers, hemp fibers, palm fibers, bamboo fibers, jute and cotton fibers.

In some examples of the presently disclosed subject matter, the solid core comprises cotton fibers.

In some examples of the presently disclosed subject matter, the solid core comprises jute fibers.

In some examples of the presently disclosed subject matter, the solid core comprises bamboo fibers.

In some examples of the presently disclosed subject matter, the solid core comprises wood fibers.

In some examples of the presently disclosed subject matter, the solid core comprises a particulate hydrocolloid.

In the context of the presently disclosed subject matter, the term “hydrocolloid” is to be understood to encompass any substance that is capable of forming a viscous, yet, non-flowing, dispersion or a non-flowing gel when mixed with water or other aqueous solutions.

In some examples of the presently disclosed subject matter, the hydrocolloid is or forms (e.g. in contact with water) a hydrogel.

In some examples of the presently disclosed subject matter, the particulate hydrocolloid comprises a polysaccharide.

In some examples of the presently disclosed subject matter, the particulate hydrocolloid comprises a polysaccharide selected from the group consisting of alginate, agar-agar, agarose, carrageenan, pectin, methylcellulose, hydroxypropyl methylcellulose (HPMC), ethylcellulose, carboxymethyl cellulose (CMC), microcrystalline cellulose, hydroxyethyl cellulose (HEC), hydroxypropyl cellulose (HPC), carboxymethyl hydroxyethylcellulose (CMHEC), carboxymethyl hydroxypropylcellulose (CMHPC), chitosan, carboxymethyl chitosan, xanthan gum, guar gum, locust bean gum, galactomannan, konjac gum, glucomannan, tara gum, gellan gum, acacia gum (Gum Arabic), curdlan, fucoidan, pullulan, hyaluronic acid and any combination of same.

It is to be appreciated that the hydrocolloids according to the present invention can be self-linked (also known, at times, as “self-cross linked”) or cross linked.

In some examples of the presently disclosed subject matter, the particulate hydrocolloid comprises a self-linked polysaccharide.

A non-limiting list of self-linked polysaccharides includes self-linked alginate, self-linked agarose, self-linked chitosan.

In some examples of the presently disclosed subject matter, the particulate hydrocolloid comprises a cross-linked polysaccharide.

A non-limiting list of cross-linked polysaccharides includes cross-linked alginate, cross-linked starch, cross-linked cellulose, cross-linked Chitosan, cross-linked Xanthan Gum, cross-linked Pectin, and cross-linked Guar Gum.

In some examples of the presently disclosed subject matter, the particulate hydrocolloid comprises or is cross-linked alginate. Cross linked alginate can be obtained using any one of a divalent or trivalent metal cation. This may include, without being limited thereto, any one or combination of Ca²⁺, Mg²⁺, Fe²⁺, Fe³⁺.

In some examples of the presently disclosed subject matter, the particulate hydrocolloid comprises or is alginate crosslinked with Ca²⁺, referred to as calcium alginate, or Ca-alginate.

In some examples of the presently disclosed subject matter, the particulate hydrocolloid comprises or is gelatin.

In some examples of the presently disclosed subject matter, the population of particles can comprise a combination of the above solid core materials.

It is to be appreciated that the particles in the population of particles can include a single solid core, or two or more solid cores held together, e.g. by the barrier coating (if present) and/or binder layer or entrapped within the scaffold.

In some examples of the presently disclosed subject matter, at least some of the particles in the population of particles include each, a single solid core.

In some examples of the presently disclosed subject matter, at least some of the particles in the population of particles include each, two or more solid cores.

In some examples of the presently disclosed subject matter, the population of particles include each, a single solid core.

In some examples of the presently disclosed subject matter, the population of particles include at least one layer of a barrier coating, enclosing or embedding at least one, at times, preferably one, solid core.

As further discussed herein, the barrier coating layer can also function as a binder layer, e.g. for associating the scaffold forming material to the solid core.

The at least one barrier coating forms a barrier for the immediate exchange between the entrapped gas and the external water. Thus, when present, the barrier coating may facilitate or contribute to the control of the gas/water exchange and thereby of the floatation/settling time.

The barrier coating is typically from a material that is water insoluble.

In some examples of the presently disclosed subject matter, the barrier coating comprises biodegradable polymers.

In some examples of the presently disclosed subject matter, the barrier coating comprises polysaccharide or polysaccharide derivative.

In some examples of the presently disclosed subject matter, the barrier coating comprises a polysaccharide or a biodegradable polysaccharide.

In some examples of the presently disclosed subject matter, the barrier coating comprises a hydrocolloid, i.e. the barrier coating is a hydrocolloid-containing coating. A non-limiting list of hydrocolloids that can form a film or layer of barrier (or, binder) coating over the solid core include any one or combination of alginate, agar-agar, agarose, carrageenan, pectin, methylcellulose, hydroxypropyl methylcellulose (HPMC), ethylcellulose, carboxymethyl cellulose (CMC), microcrystalline cellulose, hydroxyethyl cellulose (HEC), hydroxypropyl cellulose (HPC), carboxymethyl hydroxyethylcellulose (CMHEC), carboxymethyl hydroxypropylcellulose (CMHPC), chitosan, carboxymethyl chitosan, xanthan gum, guar gum, locust bean gum, galactomannan, konjac gum, glucomannan, tara gum, gellan gum, acacia gum (Gum Arabic), curdlan, fucoidan, pullulan and hyaluronic acid.

Similar to the hydrocolloids that can form the solid core, the hydrocolloid-base coating layer over the solid core can be self-linked or cross-linked.

In some examples of the presently disclosed subject matter, the coating over the solid core comprises a polysaccharide, having the same meaning as defined herein with respect to the solid core. Preferably, when also acting as a binder, the polysaccharide is a cross-linked polysaccharide.

In some examples of the presently disclosed subject matter, the barrier coating comprises one or more layers of cross-linked alginate, having the same meaning as defined herein with respect to the solid core. In some examples of the presently disclosed subject matter, the at least one layer of cross-linked alginate forms also a binder for the scaffold.

In some examples of the presently disclosed subject matter, the hydrocolloid barrier coating comprises calcium alginate, having the same meaning as defined herein with respect to the cross-linked alginate forming the solid core. In some examples of the presently disclosed subject matter, at least one layer of Ca-alginate layer forms also a binder for the scaffold.

In some examples of the presently disclosed subject matter, the hydrocolloid barrier coating comprises gelatin.

In some examples of the presently disclosed subject matter, the barrier coating can comprise hydrophobic long chain organic compounds.

In some examples of the presently disclosed subject matter, the hydrophobic long chain organic compounds are or comprise wax or wax-like substances.

In some examples of the presently disclosed subject matter, the barrier coating can comprise wax.

A non-limiting list of wax or wax-like substances that can form the barrier coating over the solid core paraffin wax, rosin wax, beeswax, carnauba wax, soy wax, candelilla wax, microcrystalline wax, montan wax, rice bran wax, ozokerite wax, lanolin wax, jojoba wax, castor wax, palm wax, tallow wax, Fischer-Tropsch wax, polyethylene wax, shellac wax, polyolefin wax and combinations thereof.

The particles can comprise one or more layers of the barrier coating, and when containing more than one layer of barrier coating, the layers can be the same or different, in composition, thickness etc., as discussed below. The plurality of layers can be achieved, for example, by stepwise coating of the particles.

In some examples of the presently disclosed subject matter, the at least one layer of barrier coating is a continuous coating over the at least one solid core or over its preceding layer of coating.

As used herein, the term “continuous coating” refers to a uniform layer of the coating material over the core without visible gaps.

In some examples of the presently disclosed subject matter, the at least one layer of barrier coating is a fragmented or discontinuous coating over the at least one solid core or over its preceding layer of coating.

As used herein, the term “segmented or discontinuous coating” refers to a non-uniform coating which is characterized by presence of gaps, interruptions, or variations in coverage, resulting in the non-uniform or fragmented appearance.

The barrier coating layer is preferably an essentially continuous coating, essentially absent of visible gaps.

In some examples of the presently disclosed subject matter, the solid core or the at least one layer of barrier coating (if there is a barrier coating), have an irregular contour. In other words, the contour is not spherical.

In some other examples of the presently disclosed subject matter, the solid core or the at least one layer of barrier coating (if present), has a round contour. A specific example for a round contour can be a spherical contour.

The population of particles disclosed herein comprise the scaffold that is configured and/or constructed to support the growth of photosynthesizing aquatic organisms.

In some examples of the presently disclosed subject matter, the scaffold is associated at least at the outer surface of the solid core or to the outer surface of the most external barrier layer and/or binder layer, if such barrier/binder layer is present.

In the context of the presently disclosed subject matter, the term “associated” or “association” refers to fixation. The fixation can be chemical or physical, depending on the entities/component being associated/connected or in contact. To this end, it is to be understood that the association can be, without being limited thereto, by any one of bonding, adhesion, entanglement, entrapment, and attachment.

In some examples of the presently disclosed subject matter, the scaffold comprises water insoluble fibers.

When referring to “fibers” it is to be understood to include any fibrous material, as known in the art. In this context, the term “fibers” also be understood to encompasses lint fibers.

As used herein the term “lint” or “lint fibers” pertains to loose or fine fibers, threads, or small fragments of material that have become detached or separated from textiles or fabrics due to wear, friction, or mechanical action. In some examples lint manifests as lightweight, entangled, and accumulative structures, comprising individual fibers or particles loosely adhering to one another.

In some examples of the presently disclosed subject mater, the water insoluble fibers are organic, non-synthetic fibers.

A non-limiting list of organic, non-synthetic fibers that can form part of the scaffold includes abaca fibers, banana fibers, bamboo fibers, broom fibers, coir fibers, cotton fibers, cannabus fibers, elephant fibers, flax fibers, hemp fibers, jute fibers, kenaf fibers, linseed fibers, oil palm fruit fibers, ramie fibers, rice husk fibers, roselle fibers, sisal fibers, sun hemp fibers, wheat fibers, wood fibers and any combination of same.

In some examples of the presently disclosed subject mater, the scaffold comprises cotton fibers.

In some examples of the presently disclosed subject mater, the scaffold comprises cannabus fibers.

In some examples of the presently disclosed subject mater, the water insoluble fibers are synthetic fibers. Examples of synthetic fibers include polyester fibers.

In some examples of the presently disclosed subject mater, the water insoluble fibers are recycled fibers, as available and known in the art.

In some examples of the presently disclosed subject mater, the scaffold comprises water insoluble porous particulate material entrapped, bound, and/or entangled within the fibers.

In some examples, the water insoluble porous particulate material is fixedly attached at least to the fibers.

In some examples, the water insoluble porous particulate material is fixedly attached to the binder and/or barrier coating layer, if present over the solid core.

In some examples, the water insoluble porous particulate material is fixedly attached to the binder and/or barrier coating layer and/or to the fibers over/external to the binder and/or barrier coating layer.

In the above and below description, when referring to the fixation of the water insoluble porous particulate material as part of the scaffold, it is to be understood to encompass any form of entrapment of the water insoluble porous particulate matter, as part of the scaffold.

The fixation of the water insoluble porous particulate material can be by the aid of the binder, as described herein.

The water insoluble particulate material forming part of the scaffold can comprise, in accordance with one example of the presently disclosed subject matter, particulate minerals and/or particulate rock.

In some examples of the presently disclosed subject matter, the water insoluble porous particulate material comprises or is a clay mineral.

In some examples of the presently disclosed subject matter, the water insoluble porous particulate material comprises or is aluminosilicate mineral.

In some examples of the presently disclosed subject matter, the water insoluble porous particulate material comprises or is carbonate mineral.

In some examples of the presently disclosed subject matter, the water insoluble porous particulate material comprises or is a mineral selected from the group consisting of montmorillonite, bentonite, halloysite, sepiolite, attapulgite and dolomite.

In some other examples of the presently disclosed subject matter, the water insoluble porous particulate material forming part of the scaffold comprises bentonite.

In some other examples of the presently disclosed subject matter, the water insoluble porous particulate material forming part of the scaffold comprises montmorillonite.

In accordance with some other examples of the presently disclosed subject matter, the water insoluble porous particulate material forming part of the scaffold can comprise particulate porous rock.

In some other examples of the presently disclosed subject matter, the water insoluble porous particulate material forming part of the scaffold is selected from the group consisting of tuff, sandstone, diatomaceous earth, shale, marl and vesicular basalt.

In some other examples of the presently disclosed subject matter, the water insoluble porous particulate material forming part of the scaffold comprises or is tuff.

In accordance with yet other examples of the presently disclosed subject matter, the water insoluble particulate material forming part of the scaffold, can comprise any combination of such minerals and rocks.

In some examples, the scaffold provides physical support for the growth of the said photosynthesizing aquatic organisms.

In some examples, the scaffold provides nutritional support for the growth of the said photosynthesizing aquatic organisms.

In some examples, the scaffold is constructed to allow growth of the organisms on and/or within the scaffold.

In its broadest context, the term “photosynthesizing aquatic organisms” denotes any aquatic primary producer.

In some examples of the presently disclosed subject matter, the organisms comprise at least one photosynthesizing microorganism.

In some examples of the presently disclosed subject matter, the organisms comprise algae.

In some examples of the presently disclosed subject matter, the organisms comprise microalgae.

In some examples of the presently disclosed subject matter, the organisms comprise at least phytoplankton.

In some examples of the presently disclosed subject matter, the organisms comprise at least phytoplankton and the scaffold supports growth of the phytoplankton thereon and/or thereby.

Thus, more specifically, the term “scaffold supporting growth of photosynthesizing aquatic organism”, or “growth scaffold” or “scaffold” refers to a framework, physical structure/substrate that provides at least physical support and a conducive environment for at least the growth/proliferation and preferably also attachment of the photosynthesizing aquatic organism.

In some examples of the presently disclosed subject matter, that scaffold comprises at least one nutrient, preferably a nutrient composition, suitable or selected for supporting growth of the photosynthesizing aquatic organism.

In the context of the presently disclosed subject matter it is to be understood that when referring to a nutrient, it is to be understood to encompass both micronutrients and macronutrients.

In the context of the presently disclosed subject matter, when referring to a nutrient, it is to be understood to encompass any state of the nutrient, be it ionic state or elemental state, even if not explicitly mentioned hereinabove or below. Thus, it is to be appreciated that when referring herein to a nutrient, it is not to be limited to a particular state.

A non-limiting list of nutrients that can be utilized for the support of growth photosynthesizing aquatic organism, in the context of the presently disclosed subject matter, includes iron (Fe), Zinc (Zn), Copper (Cu), Manganese (Mn), Molybdenum (Mo), Selenium (Se), Chromium (Cr), Cobalt (Co), Iodine (I), Fluorine (F), Magnesium (Mg), Silicon (Si), Nitrogen (N), Phosphorus (P), Sulfur(S), Strontium (Sr), Nickel (Ni), Vanadium (V) and any combination of same.

In some examples of the presently disclosed subject matter, the scaffold is supplemented with at least one nutrient.

In some examples of the presently disclosed subject matter, the scaffold is supplemented at least with iron, e.g. Fe³⁺.

In some examples of the presently disclosed subject matter, the scaffold is supplemented at least with manganese, e.g. Mn²⁺.

In some examples of the presently disclosed subject matter, the scaffold is supplemented at least with a composition comprising iron and manganese.

In some examples of the presently disclosed subject matter, the scaffold is supplemented with a composition comprising at least nitrogen containing compound, e.g. NO₃⁻.

In some examples of the presently disclosed subject matter, the scaffold is supplemented with a composition comprising at least phosphorus containing compound, e.g. PO₄³⁻.

In some examples of the presently disclosed subject matter, the scaffold is supplemented with a composition comprising at least iron, manganese, nitrogen containing compounds, and phosphorous containing compounds.

Without being bound thereto, it is assumed that the nutrients are adsorbed onto the fibers and/or onto the water insoluble particulates forming part of the scaffold.

The particles can also include a binder, in addition to, or instead of the barrier coating layer. Thus, in some examples of the presently disclosed subject matter, the at least one layer over the solid core is a hydrocolloid that can interchangeably or dually function as a barrier coating and as a binder.

In some examples of the presently disclosed subject matter, the binder is or comprises a hydrocolloid.

In some examples of the presently disclosed subject matter, the binder is or comprises a hydrocolloid of a type that can form the barrier coating.

The binder can be used for different functionalities.

In some examples of the presently disclosed subject matter, the binder is used to bind between the scaffold fibers and the outer surface of the solid core or of the barrier coating, if the latter is present in the particles.

In some examples of the presently disclosed subject matter, the binder is used to bind between the fibers forming part of the scaffold.

In some examples of the presently disclosed subject matter, the binder is used to bind between the scaffold's fibers and the water-insoluble particulate material, when also forming part of the scaffold.

In some examples of the presently disclosed subject matter, the binder is used to connect/bind between the fibers and the outer surface of the solid core or of the barrier coating and between the insoluble particulates forming part of the scaffold and the fibers forming part of the same scaffold.

In some examples of the presently disclosed subject matter, the particles comprise two or more different binders, each used to bind different components of the particles.

In some examples of the presently disclosed subject matter, the binder is a bio-based binder. Examples of bio-based binders include starch-based binders; plant-based adhesives; protein-based binders, such as gelatin; polysaccharide-based binders, such as alginate; cellulose based binders, such as lignin.

In some examples of the presently disclosed subject matter, the binder comprises alginate.

In some examples of the presently disclosed subject matter, the binder is a synthetic binder.

In some examples of the presently disclosed subject matter, the binder is a biodegradable binder.

In some examples of the presently disclosed subject matter, the scaffold is directly or indirectly associated at least at the outer surface of the solid core or of the at least one layer of barrier coating.

Direct association or linkage between the scaffold and the solid core or the barrier coating means that the scaffold material is in physical contact with the solid core or the barrier coating layer.

Indirect association or linkage between the scaffold and the solid core or the barrier coating means that there is a component interfacing between the scaffold and the solid core or the barrier coating layer. Such interfacing component is typically a binder, as disclosed herein.

The direct or indirect association can also include chemical linkage.

In some examples of the presently disclosed subject matter, there is a binder, as disclosed herein, over and/or within the at least one layer of barrier coating, and the binder binds the scaffold to the at least one layer of barrier coating.

In some examples of the presently disclosed subject matter, the scaffold is at least partially embedded within the outer surface of an existing layer of a barrier coating.

In some examples of the presently disclosed subject matter, the water-insoluble particulates forming part of the scaffold, hold the at least one nutrient. In this context, the “hold” can encompass adsorption (e.g. to the porous particulates), or embedment (e.g. into the binder material).

The adsorption or embedment of the at least one nutrient to the particles can be determined by any one of the X-ray Photoelectron Spectroscopy (XPS), Scanning Electron Microscopy with Energy Dispersive X-ray Spectroscopy (SEM-EDS), Fourier-Transform Infrared Spectroscopy (FTIR), X-ray Diffraction (XRD), Energy Dispersive X-ray Fluorescence (ED-XRF), Electron Spectroscopy for Chemical Analysis (ESCA), UV-Visible Spectroscopy, Raman Spectroscopy and Inductively Coupled Plasma Mass Spectrometry (ICP-MS).

It is noted that while it is preferable that the at least one nutrient is held by the scaffold, the particles can also hold the at least one nutrient as part of the barrier coating layer (e.g. being mixed into the material forming the barrier coating) and/or adsorbed by the solid core.

The particles of the presently disclosed population of particles hold gas that is entrapped within the at least one solid core. The gas is one having a specific gravity that is less than that of water and is present in an amount sufficient to provide floatation of the particle, at the moment the particle is brought into contact with the water.

Without being limited thereto, the gas can be any one of air, carbon dioxide (CO₂) and nitrogen (N₂).

In one specific example, the gas comprises or is air.

In one specific example, the gas comprises or is CO₂.

The amount of gas required to make a particle float in the water can be determined by mathematical and/or experimental methods.

Without being limited thereto and by way of illustration only, the volume of gas required to provide a desired positive buoyancy effect can be calculated according to the Archimedean principle.

In this context, a “positive buoyancy” can be understood to refer to the condition where the disclosed particles exhibit a density lower than that of the water in which they are distributed, resulting in an upward buoyant force exceeding the particles' gravitational weight, thereby inducing a floating or ascending behavior within the water.

In accordance with the presently disclosed subject matter, the particles, after a predefined duration following their distribution in the body of water, undergo, a transition to negative buoyancy.

In this context, a “negative buoyancy” pertains to a state in which the particles turn to have a density higher than that of the surrounding water. In this state, the upward buoyant force exerted by the water on the particles is less than the particle's gravitational weight, thereby inducing a settling or sinking or descending behavior within the water.

In some examples of the presently disclosed subject matter, the transition to a negative buoyancy is facilitated by expulsion or removal of gas from the particle, typically due to diffusion/infiltration of surrounding water into the particle.

Thus, in the context of the presently disclosed subject matter, the controlled exchange between the entrapped gas and water external to said construct can be a result of the water infiltration.

In some examples of the presently disclosed subject matter, the exchange between the entrapped gas and external water is controlled by any parameter selected from the group consisting of type material of core, dimension of core, surface area of core, porosity of core, specific gravity of core, surface energy of core, wettability of core, number of layers of said barrier coating; gas permeability of said at least one layer of barrier coating, water permeability of said at least one layer barrier coating, solubility of said at least one layer of barrier coating, thickness of said at least one layer of barrier coating, overall thickness of said barrier layer, composition of said at least one layer of barrier coating; wettability of said at least one layer of the barrier coating, overall wettability of the barrier coating, type of entrapped gas, amount of entrapped gas, water permeability of the barrier coating, water resistance of the barrier coating, gas permeability of the barrier coating, gas resistance of the barrier coating, outer surface charge, outer surface polarity and outer surface free energy.

In some examples of the presently disclosed subject matter, the exchange between the entrapped gas and external water is controlled by a combination of two or more of the above parameters.

In some examples of the presently disclosed subject matter, the controlled exchange between the entrapped gas and water external to said construct is determinable by a settling test, also known by the term “sedimentation test” or “sedimentation analysis” designed to assess the behavior of suspended solid particles in a liquid medium when subjected to gravitational forces. It involves the observation and measurement of the rate at which particles settle, % particles settling under defined test conditions. In the context of the presently disclosed subject matter, the “settling test” involves the assessment of the population of particles when suspended in water (as defined herein) including the microorganism that can grow on the scaffold, preferably, photosynthesizing microorganism/primary producers that typically harbor a photic zone of a body of water.

Without being bound by theory, it is assumed that the settling of the particles is affected by any one or combination of gas/water exchange, growth of photosynthesizing aquatic organism on the scaffold. Thus, it is to be understood that while floating at distribution over the body of water, with time, and as a result of any one or combination of gas/water exchange, growth of photosynthesizing aquatic organism on the scaffold as well as possibly other parameters, the particles will eventually settle to deeper zones of the body of water. This is one unique feature of the presently disclosed subject matter, as this will allow the capturing of carbon dioxide at the photic zone of the body of water and “removal” of the particles from the photic zone once the particles have fulfilled their purpose of carbon dioxide capturing, leaving the upper water level uncontaminated by the particles.

The amount of photosynthesizing aquatic organism grown on and/or due to the scaffold, namely, biomass, can be determined using, for example, hemocytometer or Fluorescence Activated Cell Sorter (FACS).

In some examples, the amount of biomass can be determined by the change on Total Organic Carbon (TOC) content of the particles.

Without being limited by theory, the amount of biomass can be indicative of the amount of carbon dioxide sequestration.

The particles of the presently disclosed subject matter can have any size within the range of micrometers to millimeters.

In some examples of the presently disclosed subject matter, the particles have, along their longest dimension, a size ranging from about 1 μm and about 10 millimeters; at times, between about 1 μm and about 9 mm; at times, between about 1 μm and about 8 mm; at times, between about 1 μm and about 7 mm; at times, between about 1 μm and about 6 mm; at times, between about 1 μm and about 5 mm.

The dimensions of the particles can be dictated by any one of the dimensions of the solid core, the thickness of the at least one layer of barrier coating, the thickness of the scaffold.

The dimensions of the different particle's component can be determined analytically, using, for example, any one of Scanning Electron Microscopy (SEM), Transmission Electron Microscopy (TEM), Confocal Laser Scanning Microscopy (CLSM), Sample Cross-Sectioning, Differential Weighing, Ellipsometry, Reflectance Spectroscopy, Nuclear Magnetic Resonance (NMR) Relaxometry, White Light Interferometry, X-ray Photoelectron Spectroscopy (XPS) Depth Profiling, Quartz Crystal Microbalance (QCM) with Dissipation Monitoring (QCM-D).

In some examples of the presently disclosed subject matter, the solid core dimension is in the range of between about 1 μm and about 10 millimeters; at times, between about 1 μm and about 9 mm; at times, between about 1 μm and about 8 mm; at times, between about 1 μm and about 7 mm; at times, between about 1 μm and about 6 mm; at times, between about 1 μm and about 5 mm.

In some examples of the presently disclosed subject matter, the thickness of the barrier layer coating (be it one or several layers together) is in the range of between about 1 μm and about 1 millimeter.

The population of particles can be utilized for distribution in any type of body of water. In this context, the term “water” encompasses freshwater (lakes, rivers), saltwater (oceans, seas), brackish water, saline lakes, glacier lakes, lagoons, Fjords.

As noted above, one unique feature of the presently disclosed subject matter, is that the construction and properties of the particles allow on the one hand the capturing of carbon dioxide at the photic zone of the body of water and on the other hand, “removal” of the particles from the photic zone once the particles have fulfilled their purpose of carbon dioxide capturing, leaving the upper water level uncontaminated by the particles.

In some examples of the presently disclosed subject matter, the water is saltwater.

In some examples of the presently disclosed subject matter, the water is freshwater.

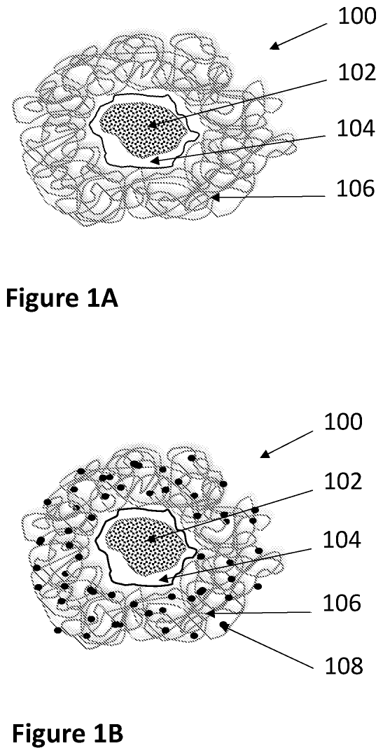

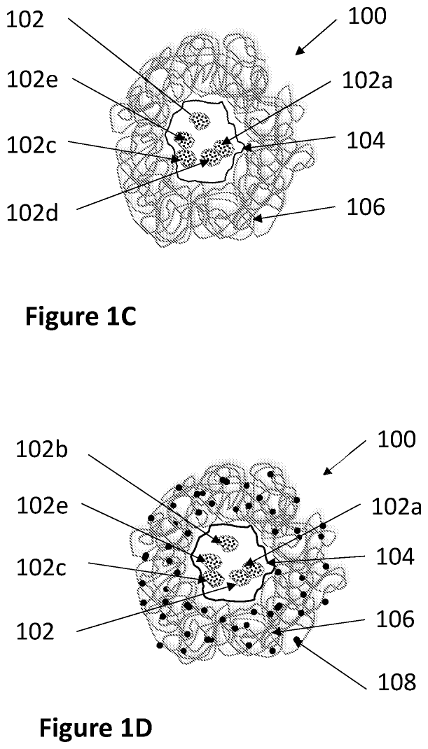

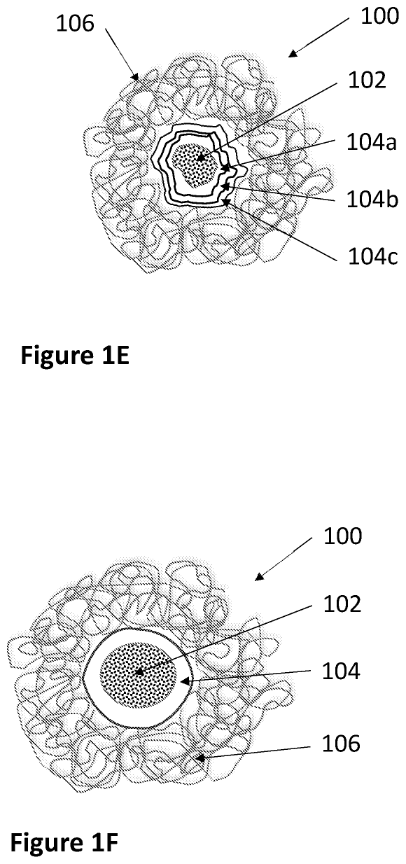

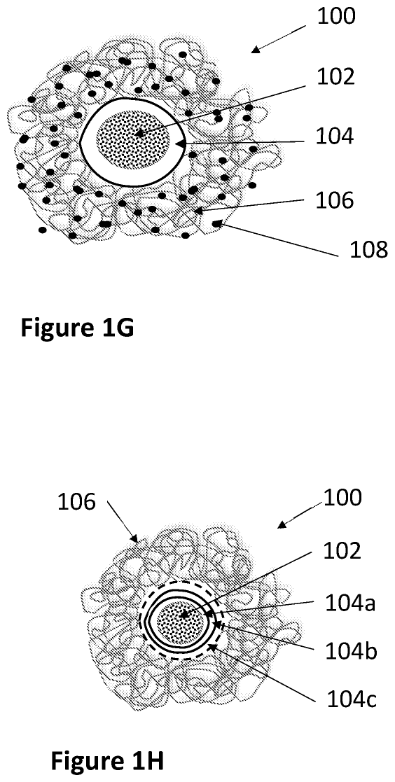

Reference is made to FIGS. 1A-1I providing schematic illustrations of different constructs according to some examples of the presently disclosed subject matter. For simplicity, FIGS. 1A-1I share the same reference numerals to identify the same components of the construct. For example, unless specifically indicated, solid core is identified by the reference number 102.

FIG. 1A provides an illustration of a particle 100 having a single solid core 102 with an irregular shape, that is coated with a single layer of a barrier coating 104. Over the barrier coating 104 there are distributed a plurality of fibers 106 forming the scaffold. The plurality of fibers may be entangled. The plurality of fibers holds growth nutrients (not illustrated).

FIG. 1B provides another illustration of a particle 100 having a single solid core 102 with an irregular shape, that is coated with a single layer of a barrier coating 104. Over the barrier coating 104 there are distributed a plurality of fibers 106 forming and attached thereto, water insoluble porous particulates 108, forming together the scaffold. The plurality of fibers may be entangled. The plurality of fibers 106 together with the porous particulates 108 hold growth nutrients (not illustrated).

FIG. 1C provides another illustration of a particle 100 having a plurality of solid cores 102a, 102b, 102c, 102d, 102e, each having an irregular shape. The plurality of solid cores 102a, 102b, 102c are embedded together within a layer of a barrier coating 104. It is noted that the plurality of solid cores need not to be spaced apart and it may occur that within a barrier coating, two solid cores are in contact, as illustrated for solid core 102a and solid core 102d and for solid core 102c and solid core 102e. Over the barrier coating 104 there are distributed a plurality of fibers 106, which may be entangled and form the growth scaffold. The plurality of fibers 106 hold growth nutrients (not illustrated).

FIG. 1D provides another illustration of a particle 100 having a plurality of solid cores 102a, 102b, 102c, 102d, 102e, each having an irregular shape. The plurality of solid cores 102a, 102b, 102c, 102d, 102e are embedded together within a layer of a barrier coating 104. Also in this illustration, at least some of the plurality of solid cores are in contact, as illustrated for solid core 102b and solid core 102c. Over the barrier coating 104 there are distributed a plurality of fibers 106, which may be entangled, and having attached to the plurality of fibers, water insoluble porous particulates 108. Fibers 106 and particulates 108 form together the growth scaffold. The plurality of fibers 106 together with the porous particulates 108 hold growth nutrients (not illustrated).

FIG. 1E provides another illustration of a particle 100 having a single solid core 102 of irregular shape. The solid core 102 is embedded in several layers of barrier coating, including most proximal layer of barrier coating 104a, sandwiched barrier coating layer 104b and a distal barrier coating layer 104c. Also in this illustration, at least some of the plurality of solid cores are in contact, as illustrated for solid core 102b and solid core 102c. Over the barrier coating 104 there are distributed a plurality of fibers 106, which may be entangled, and having attached to the plurality of fibers, water insoluble porous particulates 108. Fibers 106 and particulates 108 form together the growth scaffold. The plurality of fibers 106 together with the porous particulates 108 hold growth nutrients (not illustrated).

FIG. 1F provides another illustration of a particle 100 having a hydrocolloid solid core 102 having an essentially round shape. The hydrocolloid core 102 is embedded in a layer 104 of barrier coating or a binder for the plurality of fibers 106, which may be entangled. The plurality of fibers 106 hold growth nutrients (not illustrated).

FIG. 1G provides another illustration of a particle 100 having a hydrocolloid solid core 102 having an essentially round shape, that is coated with a single layer of a barrier coating 104. Over the barrier coating 104 there are distributed a plurality of fibers 106 forming and attached thereto, water insoluble porous particulates 108, forming together the growth scaffold. The plurality of fibers may be entangled. The plurality of fibers 106 together with the porous particulates 108 hold growth nutrients (not illustrated).

FIG. 1H provides yet another illustration of a particle 100 having a hydrocolloid solid core 102 having an essentially round shape. The core 102 is embedded in several layers of barrier coating, including most proximal layer of barrier coating 104a, sandwiched barrier coating layer 104b and a distal barrier coating layer 104c, which is difference from proximal barrier coating 104a and sandwiched barrier coating 104b. Over the barrier coating 104 there are distributed a plurality of fibers 106, which may be entangled. The plurality of fibers 106 hold growth nutrients (not illustrated).

FIG. 1I provides yet another illustration of a particle 100 having a hydrocolloid solid core 102 having an essentially round shape. The core 102 is embedded in several layers of barrier coating, including most proximal layer of barrier coating 104a, and a distal barrier coating layer 104b that is different from proximal barrier coating 104a. Over the barrier coating 104 there are distributed a plurality of fibers 106, which may be entangled and carry, attached thereto, water insoluble porous particulates 108. The plurality of fibers 106 together with the porous particulates 108 hold growth nutrients (not illustrated).

It is to be noted that in all the exemplary illustrations, entity designated 104 can exchangeable act as a binder, as a barrier coating and as both.

Some combinations of components that may be utilized in the exemplary constructions of FIGS. 1A-1I, without being limited thereto, are listed in Table 1, each possible combination in Table 1 constituting an embodiment of the presently disclosed subject matter, even if not explicitly and literally described as a combination.

TABLE 1
Combination of components

| | | Barrier/ | | |
|---|---|---|---|---|
| | | binder | | |
| FIG. | Solid core | coating | scaffold | |
| 1A | Vermiculite | Ca-alginate | Cotton fibers | |
| | and/or | | | |
| | perlite and/or | | | |
| | pumice and/or | | | |
| | natural sponge | | | |
| 1B | Vermiculite | Ca-alginate | Cotton fibers | Tuff and/or |
| | and/or | | | bentonite |
| | perlite and/or | | | and/or |
| | pumice and/or | | | montmorillonite |
| | natural sponge | | | |
| 1C | Vermiculite | Ca-alginate | Cotton fibers | |
| | and/or | | | |
| | perlite and/or | | | |
| | pumice | | | |
| 1D | Vermiculite | Ca-alginate | Cotton fibers | Tuff and/or |
| | and/or | | | bentonite |
| | perlite and/or | | | and/or |
| | pumice | | | montmorillonite |
| 1E | Vermiculite | Ca-alginate | Cotton fibers | |
| | and/or | | | |
| | perlite and/or | | | |
| | pumice | | | |
| 1F | Ca-alginate | Ca-alginate | Cotton fibers | |
| 1G | Ca-alginate | Ca-alginate | Cotton fibers | Tuff and/or |
| | | | | bentonite |
| | | | | and/or |
| | | | | montmorillonite |
| 1H | Ca-alginate | Ca-alginate | Cotton fibers | |
| 1H | Ca-alginate | Ca-alginate | Cotton fibers | |

It is to be appreciated that in the context of the presently disclosed subject matter, the population of particles can include particles of different constructs, e.g. including different solid cores material (e.g. some vermiculite, some Ca-alginate), different number of barrier coating layers (e.g. some with a single layer, some with plurality of layers), different scaffold composition (e.g. some with insoluble porous particulates, some without the porous particles), different nutrient composition, different dimensions, different type of water insoluble fibers etc. The selection of particles to form a population can be dictated by the specific needs.

The presently disclosed population of particles can have different uses.

In some examples of the presently disclosed subject matter, the population of particles are suitable for use or are used in a method for carbon dioxide sequestration, the method being as disclosed herein. Thus, the presently disclosed subject matter also discloses the use of the presently disclosed population of particles for carbon dioxide sequestration.

The presently disclosed subject matter also provides a method of producing a population of particles, the method comprising
mixing solid core material, optionally having at least one layer of barrier coating over the solid core, with a growth scaffold forming material under conditions suitable to allow association of a scaffold to at least an outer surface of the solid core;
wherein the solid core material comprises gas entrapped therein, the gas having a first specific gravity less than that of water and is in an amount sufficient to result in floatation of said particle, once the particle is brought into contact with the water;
wherein the combination of at least one solid core, the at least one layer of barrier coating, if present, and the scaffold having a second specific gravity greater than that of water; and
wherein the combination of the solid core, the at least one layer of barrier coating, if present, the scaffold and the gas is selected to provide, in the resulting particle, controlled exchange between the entrapped gas and water external to said construct, once said particle is brought into contact with water.

The presently disclosed method provides, inter alia, the presently disclosed population of particles. Thus, for the sake of simplicity, all terms and definitions provided in connection with the population of particles also apply to the presently disclosed method of producing the population of particles, mutatis mutandis.

Accordingly, in the context of the presently disclosed subject matter, the solid core material has the same meaning of the solid core forming part of the presently disclosed particles, and in the context of the presently disclosed method the solid core material is to be understood to encompass a material that allows the formation of a plurality of solid cores forming part of a population of particles, as disclosed herein.

Further accordingly, the growth scaffold forming material has the same meaning of the growth scaffold forming part of the presently disclosed particles. To this end, it is to be understood that the scaffold forming material includes water insoluble fibers as defined herein and optionally also the water insoluble porous particulate material, fixedly attached to the fibers. The fixation of the water insoluble porous particulates can be by the aid of a binder, as described herein.

Further accordingly, the entrapped gas has the same meaning as the gas forming part of the presently disclosed particles.

Further accordingly, the algae have the same meaning as the algae forming part of the presently disclosed particles.

The presently disclosed method comprises mixing the solid core material, optionally having at least one layer of barrier coating over the solid core, with a scaffold forming material under conditions suitable to allow association of a scaffold to at least an outer surface of the solid core.

In some examples of the presently disclosed subject matter, the mixing of the solid core material with the scaffold forming material is in the presence of a binder to facilitate adherence of the scaffold forming material onto the solid core.

In the context of the presently disclosed method, the binder has the same meaning as provided with respect to the presently disclosed population of particles.

In some examples of the presently disclosed subject matter, the solid core material comprises a solid core coated with at least one layer of a barrier coating.

In some examples of the presently disclosed subject matter, the at least one layer of barrier coating constitutes a binder for the scaffold forming material.

The scaffold forming material is added to the solid core coated with the barrier coating under conditions that cause binding of the scaffold forming material to the barrier coating or to the binder layer (with or without the barrier coating layer).

In some examples of the presently disclosed subject matter, to cause binding of the scaffold to the solid core, the solid core is treated with a cross linking agent. The cross-linking agent can be associated with the solid core per se or with the at least one barrier coating thereon. The scaffold forming material is mixed with a cross-linkable hydrocolloid and binding of the scaffold including the hydrocolloid is achieved by the actual cross-linking of the hydrocolloid with the cross-linking agent.

In some other examples, the binding of the scaffold forming material can be mixing with the scaffold forming material with the solid core (with or without the at least one barrier coating layer). The mixing can be, for example, by rolling the solid core (with or without the at least one barrier coating/binder layer thereon) over the scaffold forming material.

In some examples of the presently disclosed subject matter, the scaffold forming material is associated with the solid core by causing in situ enlargement of the scaffold forming material over the solid core.

In some examples of the presently disclosed subject matter, the solid core is embedded in at least one layer of barrier coating and/or is coated with a binder. This is typically, although not exclusively, prior to the association of the scaffold to the solid core.

In some examples of the presently disclosed subject matter, the method involved applying the barrier forming material over the solid core, using any coating technique known in the art. Some such techniques, include, without being limited thereto, immersion in the coating and/or binder composition, spraying of the coating and/or binder composition, utilizing Fluidized Bed Coating, Pan Coating, Hot Melt Coating, Extrusion Coating, Electrostatic Coating, Spin Coating, Fluid Coating, and combination thereof, heating of the barrier/binder material prior to application onto the solid core.

When the barrier coating layer comprises hydrocolloid, the coating can be by any technique described in the Review paper by Wei Yang et al. [Junjie Liu, Shaoxing Qu, Zhigang Suo, Wei Yang, “Functional hydrogel coatings”, National Science Review, Volume 8, Issue 2, February 2021, nwaa254, https://do aa254].

In the context of the presently disclosed method, the barrier forming material is to be understood to encompass any material that allows the formation of a barrier coating, the latter having the meaning as provided with respect to the presently disclosed population of particles.

In some examples of the presently disclosed subject matter, the embedment of the solid core in the barrier coating and/or coating of the solid core or the barrier coating layer with a binder involves self- or cross linking of the barrier coating material and/or of a binder material. Self- or cross linking is well known in the art and based on the selected material, those versed in the art would know to select the conditions to provide the desired linking.

In some examples of the presently disclosed subject matter, the barrier coating comprises a cross-linkable hydrocolloid, e.g. calcium alginate and the presently disclosed method comprises mixing the solid core material, e.g. vermiculite, with the cross-linkable hydrocolloid, e.g. the sodium alginate, following by the slow addition of a cross-linking agent, e.g. calcium chloride (the combination of sodium alginate and calcium chloride constituting the barrier forming material), to lead to the cross linking of the hydrocolloid, e.g. Ca-alginate while entrapping in the cross-linked alginate the solid core.

Alternatively, the solid core can be saturated or otherwise treated with the cross-linking agent prior to mixing the solid core with the cross-linkable hydrocolloid, and the contacting of the treated solid core with the cross-linkable hydrocolloid results of the cross-linking of the hydrocolloid while entrapping/over the solid core.

In some examples of the presently disclosed subject matter, the scaffold comprises at least one nutrient suitable for growth of the photosynthesizing aquatic organism (also referred to as aquatic primary producers). To this end, the presently disclosed method comprises mixing the scaffold forming material with a nutrient composition to allow adsorption of the nutrient composition to the scaffold forming material.

It is to be understood that the at least one nutrient and/or the nutrient composition has the same meaning as defined with respect to the presently disclosed population of particles.

In some examples of the presently disclosed subject matter, the mixing of the nutrient composition is with the scaffold forming material, e.g. the water insoluble fibers forming the scaffold. The mixing of the fibers with the nutrient composition can be before or after associating the scaffold forming material, e.g. the fibers to the solid core or to the barrier coating/binder layer, if present.

In some examples of the presently disclosed subject matter, the mixing of the nutrient composition is with the water insoluble porous particulates, either before the water insoluble porous particulates are associated to the fibers forming the scaffold or after said association.

It is to be appreciated that the water insoluble fibers and the water insoluble porous material have the same meaning as provided with respect to the population of particles.

In some examples of the presently disclosed subject matter, the mixing of the nutrient composition is with the barrier forming material, prior to applying the barrier forming material over the solid core.

In some examples of the presently disclosed subject matter, the method comprises associating (preferably, fixedly attaching) the water insoluble porous particulate material to the water insoluble fibers. The association can be before or after affixing the water insoluble fibers to the solid core or to the at least one barrier coating, if present. The association between the water insoluble fibers and the water insoluble porous particulates can be achieved with the aid of a binder as defined herein.

In some examples of the presently disclosed subject matter, the method comprises applying a binder over the solid core.

In some examples of the presently disclosed subject matter, the method comprises applying a binder over the at least one barrier coating.

In some examples of the presently disclosed subject matter, the method comprises applying a binder over the water insoluble fibers forming part of the scaffold, prior to contacting the said fibers with the water insoluble porous particulates.

In some examples of the presently disclosed subject matter, the method comprises spraying the binder.

In some examples of the presently disclosed subject matter, the method comprises immersing in a binder solution of the component that needs to hold the binder, e.g. the solid core, the solid core with the at least one barrier coating layer, the fibers forming the scaffold, the water insoluble porous particulates etc.

In some examples of the presently disclosed subject matter, the binding is a result of cross-linking. For example, the solid core or the barrier coating layer can be saturated with a cross linking agent and the contacting of the solid core with a solution of cross-linkable hydrocolloid (cross linkable with the cross-linking agent), a priori mixed with the scaffold material, will result in the distribution and fixed association of the scaffold forming material over the solid core. The cross-linkable hydrocolloid can act according to this method step the barrier coating to which the scaffold is attached, or as a binder, over the solid core or over an existing barrier coating layer.

In some examples of the presently disclosed subject matter, the method comprises actively introducing gas into the solid core. In the context of the presently disclosed subject matter the term “active introducing” is to be understood to mean applying an action that results in entrapping within the core, an amount of gas, that would not be present in the core under passive conditions.

In some examples of the presently disclosed subject matter, the active introducing involves bubbling of gas.

In some examples of the presently disclosed subject matter, the active introducing involves gas permeation.

In some examples of the presently disclosed subject matter, the active introducing involves gas injection.

In some examples of the presently disclosed subject matter, the active introducing involves gas releasing chemical reaction. Non-limiting example of gas released by a chemical reaction includes the release of CO₂ gas by the chemical decomposition of carbonate salts.

In some examples of the presently disclosed subject matter, the method comprises controlling dimension of the particles within the population of particles.

In some examples of the presently disclosed subject matter, the control of dimension of the particles can be by sieving, to select a size threshold.

In some examples of the presently disclosed subject matter, the control of dimension of the particles can be by downsizing the particles, e.g. by grinding the solid core material to a desired size, prior to applying the barrier coating (if to be present) and the scaffold forming material etc.

In some examples of the presently disclosed subject matter, the control of dimension of the particles is to a size of less than about 1 cm.

The presently disclosed subject matter also provides, in accordance with a third of its aspects, a method for carbon dioxide sequestration, the method comprising distribution a population of particles over a selected area of body of water comprising at least one photosynthesizing aquatic organism, having the meaning as provided herein, and being open to a source of carbon dioxide to be sequestered; wherein the population of particles are as defined herein with respect to the first aspect of the presently disclosed subject matter.

The presently disclosed sequestration method employs, inter alia, the presently disclosed population of particles. Thus, for the sake of simplicity, all terms and definitions provided in connection with the population of particles also apply to the presently disclosed method of carbon dioxide sequestration, mutatis mutandis.

In some examples of the presently disclosed subject matter, the sequestration method comprises receiving data relating to the selected area of body of water prior to particles distribution and determining success rate of sequestration based on the data.

According to some examples of the presently disclosed population of particles, method of producing and method of using the population of particles, involve any one of the following specific combinations, each constituting a different and independent embodiment:
vermiculite containing solid core, Ca-alginate barrier layer, scaffold comprising cotton and/or cannabus fibers.
Vermiculite containing solid core, Ca-alginate barrier layer, scaffold comprising cotton and/or cannabus fibers and tuff and/or bentonite and/or montmorillonite particulates.
Calcium alginate solid core, scaffold comprising cotton and/or cannabus fibers.
Calcium alginate solid core, scaffold comprising cotton and/or canabus fibers and tuff and/or bentonite and/or montmorillonite particulates.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to =10%.

The indefinite articles “a” and “an” as used herein in the description and in the claims, unless clearly indicated to the contrary, should be understood to mean “at least one”. It must be noted that, as used in this description and the appended claims, the singular forms “a”, “an” and “the” include plural referents unless the content clearly dictates otherwise.

The clause “and/or” as used herein in the description and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the description and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either” “one of” “only one of” or “exactly one of” “consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this description (including the Examples) and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should be understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of the presently disclosed subject matter may be presented in a range format. It should be understood that the description in range format is merely for convenience and brevity and should not be construed as an inflexible limitation on the scope of the presently disclosed subject matter. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub combination or as suitable in any other described embodiment of the presently disclosed subject matter. Certain features described in the context of various embodiments are not to be considered essential features of those embodiments, unless the embodiment is inoperative without those elements.

Various embodiments and aspects of the presently disclosed subject matter as delineated herein above and as claimed in the claims section below find experimental support in the following examples.

Disclosed and described, it is to be understood that the presently disclosed subject matter is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the presently disclosed subject matter will be limited only by the appended claims and equivalents thereof.

### LIST OF EMBODIMENTS

Some non-limiting embodiments encompassed by the present invention are defined in the following numbered clauses:
1. A population of particles, each particle comprising
   a construct comprising:
   at least one solid core,
   optionally, at least one barrier coating layer over the at least one solid core, and
   a scaffold associated at least at the outer surface of said at least one solid core or of said barrier coating, if said barrier coating is present in the construct, the scaffold being suitable for support growth of photosynthesizing aquatic organism;
   gas entrapped within said at least one solid core, the gas having a first specific gravity less than that of water and present in an amount sufficient to provide floatation of said particle, once the particle is brought into contact with the water,
   said construct having a second specific gravity greater than that of water, and
   said at least one solid core, or said barrier coating, if present in said construct, having a permeability configured to allow controlled exchange between the entrapped gas and water external to said construct.
2. The population of particles of clause 1, wherein said solid core comprises or is expanded or porous particulate.
3. The population of particles of clause 1 or 2, wherein said core comprises expanded particulate mineral.
4. The population of particles of clause 2 or 3, wherein said particulate mineral is selected from the group consisting of vermiculite (including expanded vermiculite), montmorillonite, bentonite, hectorite, saponite, kaolinite, halloysite, illite, palygorskite, sepiolite, nontronite, and any combinations of same.
5. The population of particles of clause 4, wherein said particulate mineral is expanded vermiculite.
6. The population of particles of clause 2, wherein said solid core is or comprises an expanded particulate volcanic glass.
7. The population of particles of clause 6, wherein said expanded particulate volcanic glass is or comprises expanded perlite.
8. The population of particles of clause 7, wherein said particulate volcanic glass is or comprises expanded pumice.
9. The population of particles of clause 1 or 2, wherein said solid core is a particulate organic core.
10. The population of particles of clause 9, wherein said particulate organic core is selected from the group consisting of carbon-based sponge, carbon-based foam, carbon-based fibrous material.
11. The population of particles of any one of clauses 1 to 10, comprising a single solid core, embedded within the at least one layer of barrier coating.
12. The population of particles of any one of clauses 1 to 10, each particle comprising two or more solid cores embedded within the barrier coating.
13. The population of particles of clause 1 or 2, wherein said solid core comprises a particulate hydrocolloid.
14. The population of particles of clause 13, wherein said particulate hydrocolloid comprises a polysaccharide.
15. The population of particles of clause 14, wherein said particulate hydrocolloid comprises a polysaccharide selected from the group consisting of alginate, agar-agar, agarose, carrageenan, pectin, methylcellulose, hydroxypropyl methylcellulose (HPMC), ethylcellulose, carboxymethyl cellulose (CMC), microcrystalline cellulose, hydroxyethyl cellulose (HEC), hydroxypropyl cellulose (HPC), carboxymethyl hydroxyethylcellulose (CMHEC), carboxymethyl hydroxypropylcellulose (CMHPC), chitosan, carboxymethyl chitosan, xanthan gum, guar gum, locust bean gum, galactomannan, konjac gum, glucomannan, tara gum, gellan gum, acacia gum (Gum Arabic), curdlan, fucoidan, pullulan, hyaluronic acid and any combination of same.
16. The population of particles of clause 14 or 15, wherein said particulate hydrocolloid comprises a self linked or cross-linked polysaccharide.
17. The population of particles of clause 16, wherein said particulate hydrocolloid comprises a cross-linked polysaccharide.
18. The population of particles of clause 17, wherein said particulate hydrocolloid comprises calcium alginate.
19. The population of particles of clause 13, wherein said particulate hydrocolloid comprises or is gelatin.
20. The population of particles of any one of clauses 1 to 19, comprising at least one layer of said barrier coating over the at least one solid core.
21. The population of particles of clause 20, wherein the at least one layer of barrier coating comprises or is a hydrocolloid coating.
22. The population of particles of clause 20 or 21, wherein said barrier coating comprises a hydrocolloid selected from the group consisting of alginate, agar-agar, agarose, carrageenan, pectin, methylcellulose, hydroxypropyl methylcellulose (HPMC), ethylcellulose, carboxymethyl cellulose (CMC), microcrystalline cellulose, hydroxyethyl cellulose (HEC), hydroxypropyl cellulose (HPC), carboxymethyl hydroxyethylcellulose (CMHEC), carboxymethyl hydroxypropylcellulose (CMHPC), chitosan, carboxymethyl chitosan, xanthan gum, guar gum, locust bean gum, galactomannan, konjac gum, glucomannan, tara gum, gellan gum, acacia gum (Gum Arabic), curdlan, fucoidan, pullulan, hyaluronic acid and any combination of same.
23. The population of particles of clause 21 or 22, wherein said hydrocolloid coating comprises a self linked or cross-linked polysaccharide.
24. The population of particles of clause 23, wherein said hydrocolloid coating comprises a cross-linked polysaccharide.
25. The population of particles of clause 24, wherein said hydrocolloid coating comprises calcium alginate.
26. The population of particles of clause 20, wherein said hydrocolloid coating comprises or is gelatin.
27. The population of particles of any one of clauses 1 to 20, wherein the at least one layer of barrier coating, if present, comprises a long chain organic substance.
28. The population of particles of any one of clauses 1 to 20 or 27, wherein said at least one layer of barrier coating, if present, comprises or is a wax coating.
29. The population of particles of clause 28, wherein said wax coating is selected from the group consisting of paraffin wax, rosin wax, beeswax, carnauba wax, soy wax, candelilla wax, microcrystalline wax, montan wax, rice bran wax, ozokerite wax, lanolin wax, jojoba wax, castor wax, palm wax, tallow wax, Fischer-Tropsch wax, polyethylene wax, shellac wax, polyolefin wax and combinations thereof.
30. The population of particles of any one of clauses 1 to 29, comprising two or more layers of said barrier coating, which may be the same or different.
31. The population of particles of any one of clauses 1 to 30, wherein said at least one layer of barrier coating, if present, is a continuous layer coating over said at least one solid core.
32. The population of particles of any one of clauses 1 to 31, wherein the barrier coating, if present, has an irregular contour.
33. The population of particles of any one of clauses 1 to 31, wherein the barrier coating, if present, or the solid core has an essentially round contour.
34. The population of particles of any one of claims 1 to 33, wherein said scaffold comprises a nutrient composition suitable for supporting growth of photosynthesizing aquatic organism.
35. The population of particles of clause 34, wherein said nutrient composition comprises at least one nutrient selected from the group consisting of iron (Fe), Zinc (Zn), Copper (Cu), Manganese (Mn), Molybdenum (Mo), Selenium (Se), Chromium (Cr), Cobalt (Co), Iodine (I), Fluorine (F), Magnesium (Mg), Silicon (Si), Nitrogen (N), Phosphorus (P), Sulfur(S), Strontium (Sr), Nikel (Ni), Vanadium (V) and any combination of same.
36. The population of particles of clause 34 or 35, wherein said at least one nutrient comprises at least iron.
37. The population of particles of any one of clauses 34 to 35, wherein said at least one nutrient comprises at least Manganese (Mn).
38. The population of particles of any one of clauses 1 to 37, wherein said scaffold comprises any one or combination of fibers and water-insoluble porous particulate material.
39. The population of particles of clause 38, wherein said fibers are organic fibers.
40. The population of particles of clause 39, wherein said organic fibers are non-synthetic organic fibers.
41. The population of particles of clause 39 or 40, wherein said organic fibers are selected from the group consisting of abaca fibers, banana fibers, bamboo fibers, broom fibers, coir fibers, cotton fibers, cannabus fibers, elephant fibers, flax fibers, hemp fibers, jute fibers, kenaf fibers, linseed fibers, oil palm fruit fibers, ramie fibers, rice husk fibers, roselle fibers, sisal fibers, sun hemp fibers, wheat fibers, wood fibers and any combination of same.
42. The population of particles of clause 39 or 40, wherein said organic fibers comprise cotton fibers.
43. The population of particles of clause 39, wherein said fibers comprise synthetic fibers.
44. The population of particles of clause 39, wherein said synthetic fibers comprise polyester fibers.
45. The population of particles of clause 39, wherein said fibers comprise recycled fibers.
46. The population of particles of any one of clauses 38 to 45, wherein said scaffold comprises said particulate porous insoluble material fixedly attached to said fibers.
47. The population of particles of any one of clauses 1 to 46, comprising a binder.
48. The population of particles of clause 47, wherein said binder is a bio-based binder and/or biodegradable binder.
49. The population of particles of clause 47, wherein said binder is a synthetic binder.
50. The population of particles of any one of clauses 47 to 49, whenever dependent on claim 38, wherein said binder binds between any one or combination of (i) said fibers of the scaffold and said outer surface of the particulate (ii) fibers of the scaffold; (iii) fibers of said scaffold and water-insoluble porous particulate material, if present in the scaffold.
51. The population of particles of any one of clauses 38 to 50, whenever dependent on claim 38, wherein said water-insoluble porous particulate material comprises minerals and/or particulate rock.
52. The population of particles of claim 51, wherein said water-insoluble porous particulate material comprises a clay mineral, aluminosilicate mineral and/or a carbonate mineral.
53. The population of particles of clause 52, wherein said water-insoluble porous particulate material is a mineral selected from the group consisting of zeolites, bentonite, montmorillonite halloysite, sepiolite, attapulgite and dolomite.
54. The population of particles of clause 52 or 53, wherein said water insoluble porous particulate matter comprises bentonite and/or montmorillonite.
55. The population of particles of claim 51, wherein said insoluble material comprises particulate rock.
56. The population of particles of clause 55, wherein said water-insoluble porous particulate material is a particulate rock selected from the group consisting of tuff, sandstone, diatomaceous earth, shale, marl and vesicular basalt.
57. The population of particles of clause 52 or 56, wherein said water-insoluble porous particulate material is tuff.
58. The population of particles of any one of clauses 1 to 57, whenever dependent on claim 34 and claim 38, wherein said water-insoluble porous particulate material holds said nutrient composition.
59. The population of particles of any one of clauses 1 to 58, comprising said at least one layer of said barrier coating, and wherein said scaffold is directly or indirectly, associated at least at the outer surface of said at least one layer of barrier coating.
60. The population of particles of claim 59, wherein said barrier layer is a binder layer or said particles comprises a binder over and/or within said at least one layer of barrier coating and said binder binds said scaffold to the solid core.
61. The population of particles of any one of clauses 1 to 60, comprising at least one layer of said barrier coating and said scaffold is at least partially embedded within said outer surface of the barrier coating.
62. The population of particles of any one of clauses 1 to 61, wherein said scaffold is directly or indirect chemical linked to the outer surface of the solid core or to the outer surface of the at least one layer of barrier coating, if present in the particle.
63. The population of particles of any one of clauses 1 to 62, wherein said gas is selected from the group consisting of air and CO₂.
64. The population of particles of any one of clauses 1 to 63, wherein said exchange between the entrapped gas and water external to said construct is controlled by a combination of at least one selected from the group consisting of material of core, dimension of core, surface area of core, porosity of core, specific gravity of core, surface energy of core, wettability of core, number of layers of said barrier coating; gas permeability of said at least one layer of barrier coating, water permeability of said at least one layer barrier coating, solubility of said at least one layer of barrier coating, thickness of said at least one layer of barrier coating, overall thickness of said barrier layer, composition of said at least one layer of barrier coating; wettability of said at least one layer of the barrier coating, overall wettability of the barrier coating, type of entrapped gas, amount of entrapped gas, water permeability of the barrier coating, water resistance of the barrier coating, gas permeability of the barrier coating, gas resistance of the barrier coating, outer surface charge, outer surface polarity, outer surface free energy.
65. The population of particles of any one of clauses 1 to 64, wherein said controlled exchange between the entrapped gas and water external to said construct is determinable by a settling test whereby at least one of particles' settling rate, % particles settling, amount of particles settling under defined conditions is determined.
66. The population of particles of any one of clauses 1 to 65, wherein said water is saltwater.
67. The population of particles of any one of clauses 1 to 65, wherein said photosynthesizing aquatic organism comprises microalgae.
68. The population of particles of any one of clauses 1 to 67, wherein said particles have an average size between the micrometer range and millimeter range.
69. A method of producing a population of particles, the method comprising
mixing solid core material, optionally having at least one layer of barrier coating over the solid core, with a scaffold forming material under conditions suitable to allow association of the scaffold material to at least an outer surface of the solid core;
wherein said solid core material comprises gas entrapped therein, the gas having a first specific gravity less than that of water and is in an amount sufficient to result in floatation of said particle, once the particle is brought into contact with the water;
wherein the combination of at least one solid core, said at least one layer of barrier coating, if present, and the scaffold having a second specific gravity greater than that of water; and
wherein the combination of the solid core, the at least one layer of barrier coating, if present, the scaffold and the gas is selected to provide, in the resulting particle, controlled exchange between the entrapped gas and water external to said construct, once said particle is brought into contact with water.
70. The method of clause 69, comprising mixing said solid core with a barrier material under conditions that result in coating, in a single particle, of one or more solid cords with at least one layer of barrier coating.
71. The method of clause 70, wherein said conditions comprises formation of a hydrocolloid embedding one or more solid cores.
72. The method of any one of clauses 69 to 71, wherein said solid core comprises or is expanded or porous particulate.
73. The method of clause 72, wherein said core is or comprises an expanded particulate mineral.
74. The method of clause 73, wherein said expanded particulate is expanded vermiculite.
75. The method of clause 73, wherein said core is or comprises an expanded particulate volcanic glass.
76. The method of clause 75, wherein said expanded particulate volcanic glass is or comprises expanded perlite or pumice.
77. The method of clause 72, wherein said solid core comprises or is a particulate organic core.
78. The method of any one of clauses 69 to 71, wherein said solid core comprises a particulate hydrocolloid.
79. The method of clause 78, wherein said particulate hydrocolloid comprises a polysaccharide or gelatin.
80. The method of claim 79, wherein said polysaccharide is self-linked or cross-linked polysaccharide.
81. The method of any one of clauses 69 to 80, wherein said scaffold forming material comprises any one or combination of fibers and water-insoluble porous particulate material.
82. The method of clause 81, wherein said fibers are organic fibers.
83. The method of clause 82, wherein said organic fibers are selected from the group consisting of abaca fibers, banana fibers, bamboo fibers, broom fibers, coir fibers, cotton fibers, cannabus fibers, elephant fibers, flax fibers, hemp fibers, jute fibers, kenaf fibers, linseed fibers, oil palm fruit fibers, ramie fibers, rice husk fibers, roselle fibers, sisal fibers, sun hemp fibers, wheat fibers, wood fibers and any combination of same.
84. The method of clause 82 or 83, wherein said organic fibers comprise cotton fibers.
85. The method of clause 81, wherein said fibers comprise synthetic fibers.
86. The method of clause 85, wherein said fibers are synthetic are polyester fibers.
87. The method of any one of clauses 69 to 86, comprising supplementing at least said scaffold with a nutrient composition.
88. The method of clause 87, wherein said supplementing comprises contacting said scaffold material with a solution of said nutrient composition, suitable for supporting growth of algae, said contacting being prior to or after mixing the scaffold material with the solid core.
89. The method of clause 87 or 88, wherein said nutrient composition comprises at least one nutrient selected from the group consisting of iron (Fe), Zinc (Zn), Copper (Cu), Manganese (Mn), Molybdenum (Mo), Selenium (Se), Chromium (Cr), Cobalt (Co), Iodine (I), Fluorine (F), Magnesium (Mg), Silicon (Si), Nitrogen (N), Phosphorus (P), Sulfur(S), Strontium (Sr), Nikel (Ni), Vanadium (V) and any combination of same.
90. The method of any one of clauses 87 to 89, wherein said nutrient composition comprises at least Fe and/or Mn.
91. The method of any one of clauses 69 to 90, whenever dependent on claim 81, comprising fixedly attaching said water insoluble porous material to said fibers and/or to said barrier coating.
92. The method of clause 88, wherein said water insoluble porous material is selected from the group consisting of minerals and/or particulate rock.
93. The method of clause 92, wherein said water-insoluble porous particulate material comprises a clay mineral, aluminosilicate mineral and/or a carbonate mineral.
94. The method of clause 93, wherein said water-insoluble porous particulate material is a mineral selected from the group consisting of zeolites, bentonite, halloysite, sepiolite, attapulgite and dolomite.
95. The method of clause 92, wherein said insoluble material comprises particulate rock.
96. The method of clause 95, wherein said water-insoluble porous particulate material is a particulate rock selected from the group consisting of tuff, sandstone, diatomaceous earth, shale, marl and vesicular basalt.
97. The method of clause 96, wherein said water insoluble porous material comprises tuff.
98. The method of any one of clauses 81 to 97, comprising adsorbing said nutrient composition onto said water insoluble porous material.
99. The method of any one of clauses 69 to 98, comprising contacting said solid core material with a barrier composition suitable for forming said at least one layer of barrier coating over said solid core.
100. The method of clause 99, wherein said barrier composition comprises a hydrocolloid composition.
101. The method of clause 100, wherein said hydrocolloid composition comprises a hydrocolloid selected from the group consisting of alginate, agar-agar, agarose, carrageenan, pectin, methylcellulose, hydroxypropyl methylcellulose (HPMC), ethylcellulose, carboxymethyl cellulose (CMC), microcrystalline cellulose, hydroxyethyl cellulose (HEC), hydroxypropyl cellulose (HPC), carboxymethyl hydroxyethylcellulose (CMHEC), carboxymethyl hydroxypropylcellulose (CMHPC), chitosan, carboxymethyl chitosan, xanthan gum, guar gum, locust bean gum, galactomannan, konjac gum, glucomannan, tara gum, gellan gum, acacia gum (Gum Arabic), curdlan, fucoidan, pullulan, hyaluronic acid and any combination of same.
102. The method of clause 100 or 101, wherein said hydrocolloid composition comprises a cross-linkable hydrocolloid and said method comprises mixing said hydrocolloid composition and said solid core with a hydrocolloid cross-linking agent.
103. The method of clause 102, wherein said hydrocolloid composition comprises alginate and said cross linking agent is calcium.
104. The method of clause 103, wherein said barrier composition comprises wax.
105. The method of clause 99, wherein said barrier composition comprises a biodegradable organic substance.
106. The method of any one of clauses 99 to 105, comprises creating two or more layers of barrier coating over said solid core.
107. The method of any one of clauses 69 to 106, comprises applying a binder within and/or over said at least one layer of barrier coating, if said at least one layer of barrier coating is present, or over said solid core, prior to or concomitant with association of the scaffold.
108. The method of clause 107, comprising mixing said binder with said nutrient composition prior to applying said binder.
109. The method of clause 107 or 108, wherein said applying of the binder is by spraying and/or immersing.
110. The method of any one of clauses 107 to 109, wherein said binder is any one or combination of bio-based and biodegradable binder.
111. The method of any one of clauses 107 to 110, wherein said binder is a synthetic binder.
112. The method of any one of clauses 107 to 111, comprising applying said binder to result in any one or combination of binding between (i) said fibers of said scaffold and said outer surface (ii) fiber to fiber within said fibers of said scaffold; (iii) fibers of said scaffold and water-insoluble porous particulate material, if present at said scaffold.
113. The method of any one of clauses 69 to 112, comprising active introduction of said gas into said solid core.
114. The method of clause 113, wherein said active introduction comprises any one of bubbling of the gas, gas permeation, gas releasing chemical reaction.
115. The method of any one of clauses 69 to 114, wherein said gas is selected from the group consisting of air and CO₂.
116. The method of any one of clauses 69 to 115 whenever dependent on claim 81, comprising attaching said water insoluble porous material to said fibers.
117. The method of clause 116, wherein said attaching comprises combining said fibers with a binder and mixing the fibers with the water insoluble porous material.
118. The method of any one of clauses 69 to 117, comprising controlling dimensions of said particles in said population.
119. The method of clause 118, wherein said controlling dimensions of the particles is by selecting particles within a specific size or size range.
120. The method of clause 119, comprising selecting particles having a size below 1 cm.
121. A method for carbon dioxide sequestration, comprising distribution a population of particles over a selected area of body of water comprising at least one photosynthesizing aquatic organism and being open to a source of carbon dioxide to be sequestered; wherein said population of particles comprise a construct comprising:
   at least one solid core,
   optionally, at least one barrier coating layer over the at least one solid core, and
   a scaffold associated at least at the outer surface of said at least one solid core or of said barrier coating, if said barrier coating is present in the construct, the scaffold being suitable for support growth of the photosynthesizing aquatic organism;
   gas entrapped within said at least one solid core, the gas having a first specific gravity less than that of water and present in an amount sufficient to provide floatation of said particle, once the particle is brought into contact with the water;
   said construct having a second specific gravity greater than that of water; and
   said at least one solid core, or said barrier coating, if present in said construct, having a permeability configured to allow controlled exchange between the entrapped gas and water external to said construct.
122. The method of clause 121, comprising receiving data relating to said selected area of body of water prior to said distribution, and determining success rate of sequestration based on said data.
123. The method of clause 121 or 122, comprising actuating said distribution based on said received data.
124. The method of any one of clauses 121 to 123, wherein said population of particles is as defined in any one of clauses 1 to 68 or as obtained by the method of any one of clauses 69 to 120.

### DESCRIPTION OF NON-LIMITING EXAMPLES

### Example 1—Alginate-Coated Vermiculite

### Effect of Alginate Concentration on Duration of Particle Floatation

### Materials:

Vermiculite SA-Dry expanded Vermiculite was obtained from Sigma Aldrich (Catalog No. Z765422)
Alginate-Sodium Alginate was obtained from Sigma Aldrich (Catalog No. W201502)
CaCl₂·7H₂O was obtained from Romical (Catalog No. 433381)

### Method

Dry expanded vermiculite, (sieved size >500 μm) was coated with sodium alginate using three different alginate concentrations: 0.5%, 2% and 4% and 0.5M CaCl₂ solution adjusted with 1M HCl solution to pH-3. Each tested group (including 5 gr vermiculite particulates) was placed in 100 ml beaker and the beaker was filled with CaCl₂) solution. The samples were then filtered to remove excess CaCl₂) and thereby obtain the Ca-washed vermiculite.

For cross linking, Na alginate solution (~150 ml) was stirred vigorously (0.5% and 2% were stirred at 1000 rpm, 4% was stirred at 1030 rpm). The Ca-washed vermiculite was then added gradually and mixed into the Na alginate solution and left in the alginate for 1 minute to thereby form a Ca-alginate coating over vermiculate particulates.

The Ca-alginate coated vermiculite was then washed with double distilled water (DDW) to remove excess alginate solution. Weight of each sample was then recorded, as detailed in Table 2.

TABLE 2
Vermiculite % weight change after alginate coating

| No. | Alginate concentration* | % Weight change |
|---|---|---|
| 1 | 0.5% | 600% |
| 2 | 2% | 770% |
| 3 | 4% | Not available |

*Concentration in the alginate solution used for coating

The particles' duration of floating/sinking was evaluated in filtered and unfiltered seawater. Filtered seawater was obtained by filtering in 0.22 μm filter. For each particle type, three replicates were measured. As control groups, the floating/sinking of vermiculite (with no alginate coating) in filtered and unfiltered seawater were used.

Specifically, the test samples were placed in a container (100 ml) including seawater (50 ml, filtered or unfiltered) and top view photographs of the containers' surface were taken.

The proportion of floating particles was estimated by analyzing the surface area they covered at various sampling times compared to the initial coverage (to).

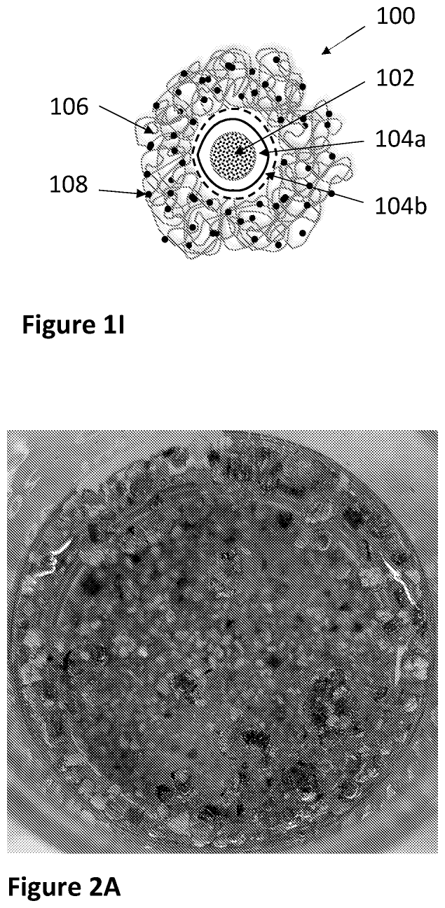

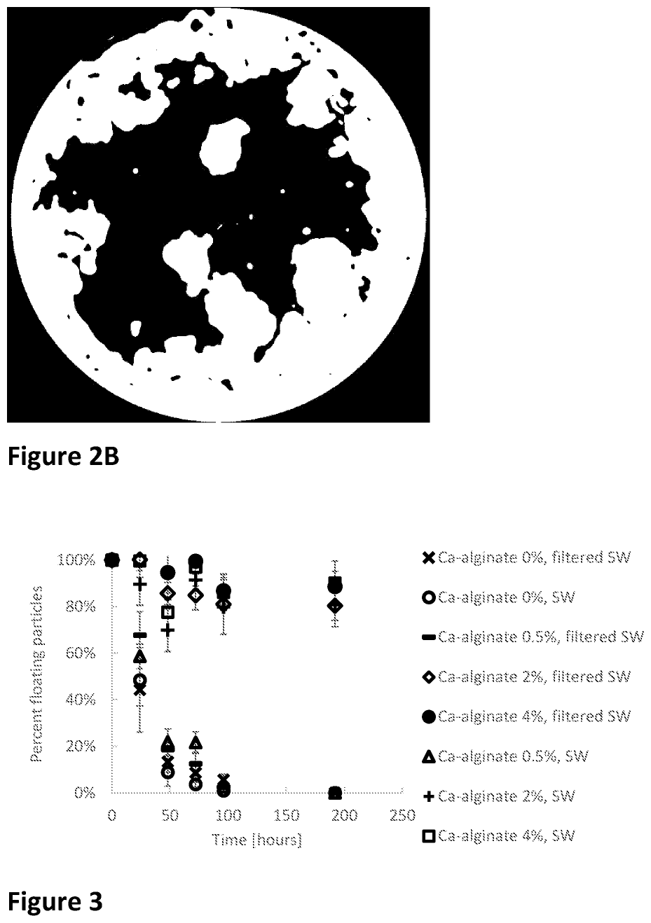

Surface coverage percentage was determined using Python version 3.11 for image processing. Top view photographs were taken for each sample (FIG. 2A). The image underwent sequential processing steps, including grayscale conversion, sharpening, gradient magnitude calculation (smoothing scale 1), and Gaussian blur (sigma radius 10).

Subsequently, a binary image was created using a threshold of 100 and 255 (FIG. 2B). Coverage percentage inside the circle was calculated by distinguishing black (seawater) and white (particle coverage) pixels.

### Results

FIG. 3 shows that increasing the thickness of Ca-alginate coating on vermiculite leads to longer floating times, which indicates that by controlling thickness of the barrier coating layer, it is possible to control the floatation time of the particles (and in other words, the exchange rate between the external water and the entrapped gas). There were notable differences between uncoated vermiculite (0% alginate) and vermiculite coated with 2% and 4% alginate, confirming that the barrier coating is significant for the control of floatation, when using a solid core that is a mineral, rock or volcanic glass based (not hydrocolloid based).

Within 24 hours, floating particles of uncoated vermiculite (control samples) covered less than 50% of the water's surface in the containers and they all sank (settled) after 8 days. In contrast, with 2% and 4% alginate-coated vermiculite over 70% of the water surface was covered by the floating particles from 24 hours to 8 days of incubation (see FIG. 3). This further confirms the need for the barrier coating to control the water/gas exchange and the floating/settling rate.

Upon coating with a 0.5% alginate solution, vermiculite exhibited significantly shorter floating times (as compared to the 2% and 4% alginate-coated ones), which was similar to uncoated vermiculite (control samples).

The type of seawater used (raw seawater or filtered seawater) did not affect the floating time, suggesting that the particles are applicable for a variety of water types.

### Effect of Number of Coating Layers

### Method

The multilayer coating process on vermiculite followed the same protocol as described hereinabove for a single layer coating, using a 0.5% alginate solution. Initially, the first layer was applied, followed by particles wash, and then a second layer was applied, continuing in this manner for subsequent layers. The last layer of alginate was washed with CaCl₂) solution to strengthen the outer coating layer.

The Ca-Alginate coated vermiculate particles were then washed with double distilled water (DDW) to remove excess alginate solution.

The particles' duration of floating/sinking was evaluated in unfiltered seawater according to the protocol described hereinabove.

### Results

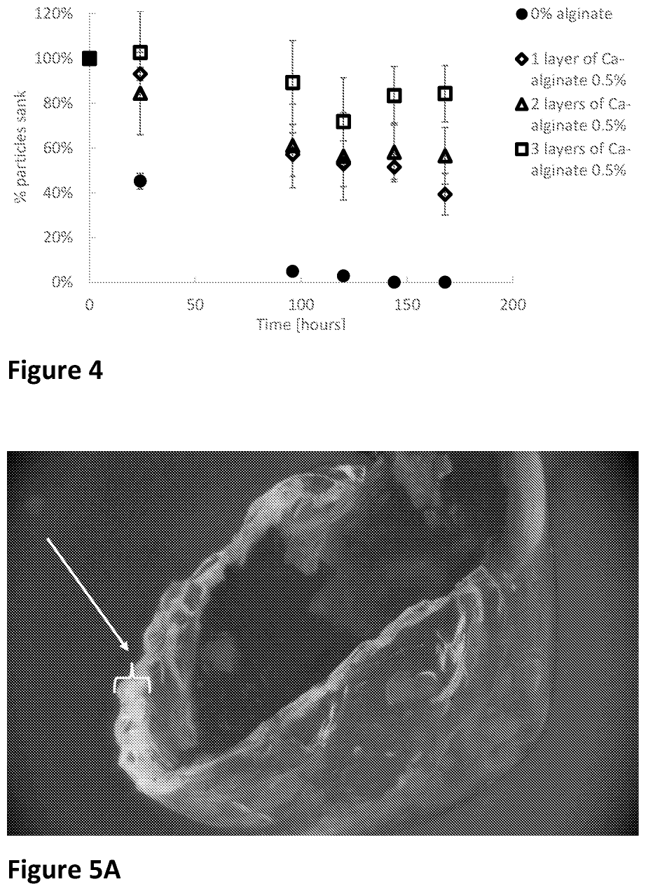

FIG. 4 shows that the vermiculite coated with multiple layers of alginate displayed a greater percentage of floating particles as the number of coating layers (and concomitantly layer thickness) increased. This trend persisted over the course of a week of incubation in seawater.

Vermiculite particulates coated with 1, 2, and 3 layers of Ca-alginate remained stable (i.e. 3 repeated coating and washing steps), with more than 50% of the particulates floating for an entire 6-day incubation period.

Furthermore, as the number of coating layers decreased, particles coated with alginate exhibited a reduction in the percentage of floating particles. This means that there is a correlation between the thickness and/or number of layers of barrier coating, and the % floatation. This is further supported by the fact that the majority of uncoated vermiculite particles sank within 24 hours (quick settling).

### Effect of Wetting Mechanism

Fluorescein, a fluorescent tracer, was employed to monitor the wetting process of the particle flotation core. This can be a tool to determine the mechanism of exchange between the entrapped gas and the external water.

### Method

Uncoated and Ca-alginate coated vermiculite particulates were prepared as described in Example 1 (0.5%, 2% and 4% Ca-alginate single layer coating) and immersed in 50 ml Falcon tubes filled with a 0.01 nM fluorescein sodium salt solution (Catalog No. F-6377, Sigma Aldrich) for a period of 7 days.

Sampling encompassed the retrieval of both floating and sunken particles from the tubes, followed by their examination under a fluorescence microscope.

### Results:

The presence of fluorescent dye in various regions of the particles indicated wetting as fluorescein solution penetrated the external surface of the particle.

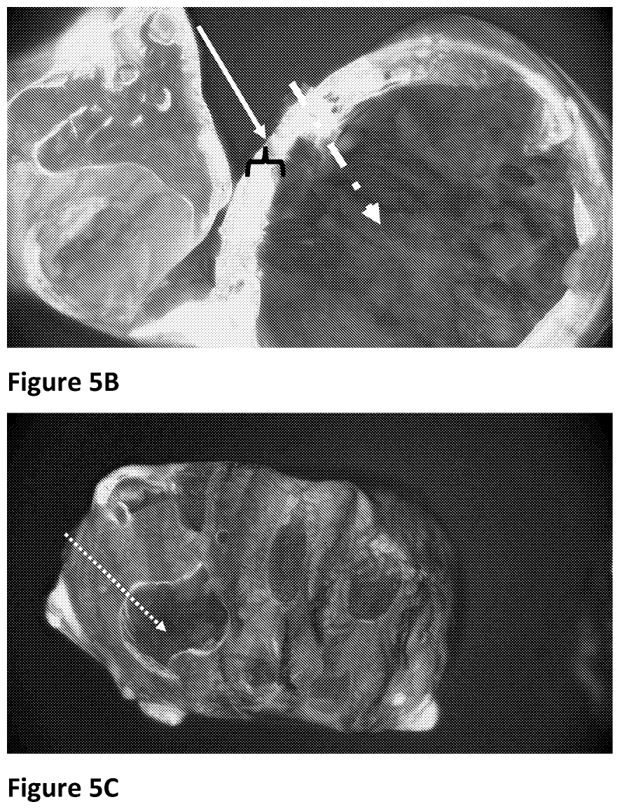

In the case of the sample taken with floating particles, the fluorescent dye was observed either exclusively on the external alginate layer (FIGS. 5A-5B) or within the coating layer itself, situated between the vermiculite particles and within the vermiculite layers (FIG. 5C).

Specifically, FIG. 5A presents a cross-section of a coated particle, highlighting the fluorescent dye exclusively within the alginate coating layer (the fluorescent dye area is marked by a full arrow). In FIG. 5B, a cross-section of a particle revealed the dry, non-dyed internal part (appears as dark inside, marked by the dashed-line arrow) juxtaposed with the dyed external layer (visible on the left side of the image, marked by a full arrow). Furthermore, FIG. 5C displays a dyed particle with entrapped air bubbles located within the coating (a representative bubble being marked by a dotted arrow).

Notably, these particles remained floating/buoyant as long as air bubbles remained trapped within the coating or the expanded mineral.

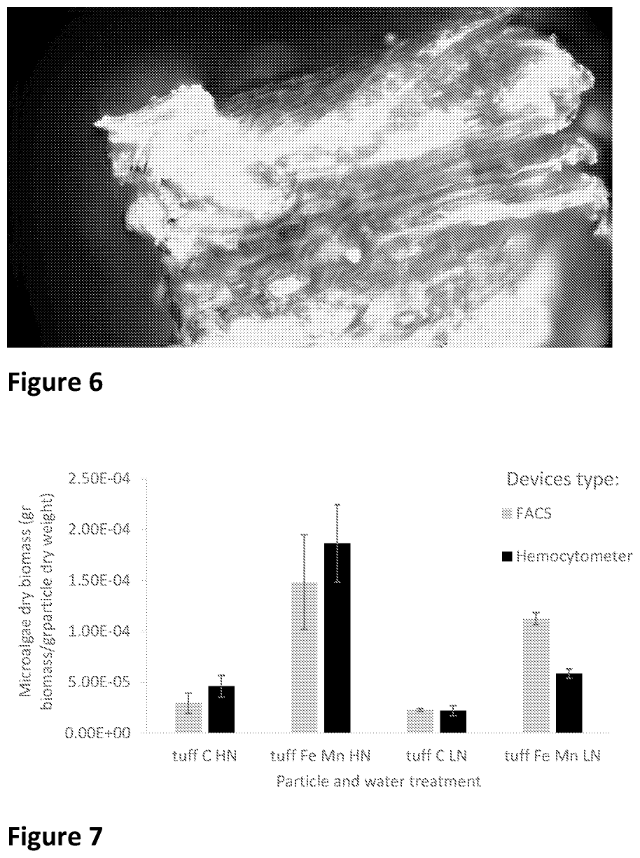

Sunken particles exhibited the fluorescent dye both in the external coating and within the spaces between the vermiculite and layers. FIG. 6 shows a vermiculite particulate from particles that has settled, and after removing the Ca-alginate barrier layer. The vermiculite particle in FIG. 6 clearly shows to hold the fluorescent dye: the light areas of the vermiculite are the interlayer spacing of the particulate, which would have been dark, in the absence of the dye.

No discernible difference in this pattern was observed between particles sinking within 1-2 days and those sinking within 5-7 days, meaning that the assumption that the settling of the particles is due to water penetration and thereby gas/water exchange is experimentally supported and further that this can be controlled, inter alia, by barrier coating properties.

### Example 2—Microalgae Growth on Solid Carrier

### Growth of Microalgae on Cotton or Tuff

### Materials:

Cotton fibers—clear fibers for cosmetic use (average fiber diameter 9.95 micron) were obtained from a pharmacy,
Tuff—fraction 500-1000 micron were obtained from Hermonit quarry, Israel.
Microalgae—Laboratory experiments were conducted with the Bacillariophyceae Phaeodactylum tricornutum strain UTEX 640 from the UTEX Culture Collection of Algae at UT-Austin
NaNO₃—purchased from Romical (catalog No. 481757).
NaH₂PO₄—purchased from Romical (catalog No. 480141).
MnCl₂·4H₂O-obtained from Sigma Aldrich (catalog No. M3634).
FeCl₃ anhydrous—obtained from Sigma Aldrich (catalog No. 908908).

### Method:

The growth of microalgae on cotton fibers or tuff particulates within seawater media was assessed. To this end, cotton fibers or tuff particulates, As Is or adsorbed with Fe+Mn, were used as the microalgae growth support scaffold.

Adsorbing Fe and Mn on tuff particulates was achieved by agitation of tuff particulates in a solution of Fe³⁺ (2 mM) and Mn²⁺ (0.2 mM) overnight and then washed in seawater three times before using for growth exper4iments.

The adsorption of iron to the cotton fibers was discernible through a change in the coloration of the fibers from white to orange-brown rust-like color.

Seawater salinity was 35 ppt and conductivity was 46 μS.

To support microalgae growth, the seawater medium was supplemented with nutrients including 25 μM NaNO₃ and 2.5 μM NaH₂PO₄ in the low nutrient treatment (SW LN), or 200 μM NaNO₃ and 20 μM KH₂PO₄ in the high nutrient treatment (SW HN).

The seawater with low nutrients (SW LN) and high nutrients (SW HN) media were then inoculated with microalgae, and cultivated for 10-14 days, until the microalgae reached a stationary phase. The stationary phase inoculum was counted using a hemocytometer and inoculated in both SW LN medium and SW HN medium at the experiment initiation. The initial cell concentration was 2.5×10⁵ cells/mL.

Untreated tuff or cotton without micronutrients (Fe+Mn) were used as controls. The samples were cultivated in the different seawater media and at different solid to liquid ratios, while rotating at 75 rpm. Table 4 provides the different test groups (n=3) and the respective conditions.

TABLE 4
Effect of nutrient on algae growth

| | | | | | Solid to |
|---|---|---|---|---|---|
| Test | | Fe + | Micronutrient | Volume | liquid ratio |
| group | Substrate | Mn | concentration | (mL) | (_{g}DW L⁻¹) |
| 1 | Tuff | — | HN | 20 | 92 |
| 2 | Tuff | — | LN | 20 | 92 |
| 3 | Tuff | ✓ | HN | 20 | 92 |
| 4 | Tuff | ✓ | LN | 20 | 92 |
| 5 | Tuff | — | HN | 50 | 36.8 |
| 6 | Tuff | ✓ | HN | 50 | 36.8 |
| 7 | Cotton | — | HN | 20 | 6 |
| 8 | Cotton | — | LN | 20 | 6 |
| 9 | Cotton | ✓ | HN | 20 | 6 |
| 10 | Cotton | ✓ | LN | 20 | 6 |

Sampling was preformed 3 times a week, with 2 days intervals.

Microalgae dry biomass assessment involved several steps. First, microalgae concentrations in three inoculums were determined through hemocytometer counting. Next, three micro glass fiber filters were weighed before filtering 5 mL of inoculum. The microalgae remaining on the filter surface area were dried in an oven at 105 °C for 1 hour. Subsequently, the dry filter and microalgal biomass were re-weighed after the drying process. The difference between the weights before and after filtration was divided by the total microalgal cells filtered.

Based on the results, the dry weight of a single microalgal cell was determined to be 55.15±3.40 pg based on three replicates (n=3), serving as the basis for dry biomass calculations in this experiment.

Alternatively, microalgae dry biomass was determined by Fluorescence-activated cell sorting (FACS) analysis which was conducted as follows: Liquid samples, each containing 0.85 mL, were placed in cryogenic vials and supplemented with 3 μL of 50% w/w glutaraldehyde. After brief vortexing, the vials were rapidly frozen in liquid nitrogen and stored at −80 °C. Prior to analysis, the samples were thawed at room temperature for 1 hour. Each vial's total sample volume was adjusted to 0.8 mL, and analysis involved assessing 50 μL from each vial. The FACS analysis was based on chlorophyll-a autofluorescence which was measured with an intensity reading at 660 nm (green emission) using an excitation wavelength of 488 nm, along with forward scatter data.

### Results:

### Attachment and Growth of Microalgae on Tuff

From day zero until the 15th day of the experiment, Fe+Mn-supplemented tuff particulates in high nutrients (HN) medium have shown significantly higher microalgae dry biomass than Fe+Mn-supplemented tuff particles in low nutrient (LN) medium. The highest microalgae dry biomass content was measured on Fe+Mn-supplemented tuff particulates in the HN medium on day 11 with 3.1×10⁻⁴ gr biomass/gr particle dry weight.

The highest dry algal biomass differences between Fe+Mn-supplemented tuff in high nutrients (HN) medium to Fe+Mn-supplemented tuff in low nutrients (LN) medium, control in HN medium, and control in LN medium were observed on days 4, 11 and 13, and were 7.31, 6.13 and 10.49 times higher, respectively.

From days 6 to 11, Fe+Mn-supplemented tuff particulates in LN medium treatment have significantly accumulated microalgae dry biomass, peaking at 1.56×10-4 gr biomass/gr particle dry weight on day 11. The greatest difference between Fe+Mn-supplemented tuff particulates in LN medium to control HN medium and to control LN medium were observed on days 11 and 13, and were 3.08 and 4.16 times higher, respectively.

The greatest microalgae dry biomass of Fe+Mn-supplemented tuff particulates in HN medium and Fe+Mn-supplemented tuff particulates in LN medium was observed on day 11. However, Fe+Mn-supplemented tuff particulates in HN medium still showed 1.98 times higher gr biomass/gr particle dry weight than in LN. On day 13, the microalgae dry biomass measured was lower than on day 11, except for control HN medium.

To validate the measurements, samples from day 6 were submitted to FACS analysis. FIG. 7 shows the microalgae dry biomass as a function of nutrient content on Tuff containing scaffold (the Tuff particulates as described above), plotted using FACS (grey) or hemocytometer (black) devices. Results show the same significant differences between treatments (p<0.05) and no significant differences between measuring devices (p=0.8998) (except on Fe- and Mn-enriched tuff particles in low nutrients medium). The results show that when supplemented with Fe and Mn, there is a significant increase in the total dry biomass, and further provides validation for the algae measuring method.

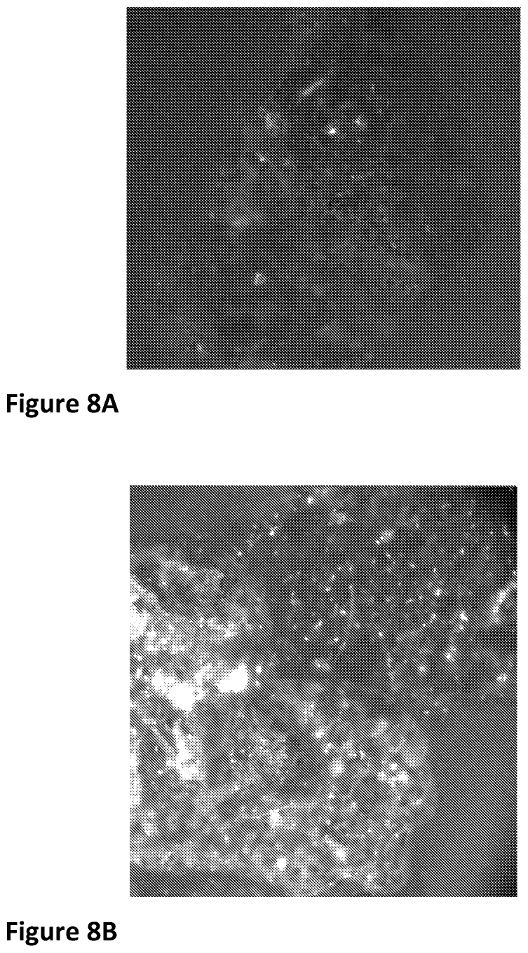

Furthermore, visual differences were observed throughout the experiment, and a higher number of algae were attached to Fe+Mn-supplemented tuff particulates in HN medium. FIGS. 8A-8B show images of attached and fluorescent microalgae (bright white spots, some marked by the white arrows) on the tuff particulates, taken on day 11. Specifically, FIG. 8A shows control tuff particulates in high nutrients medium, and FIG. 8B shows Fe+Mn-supplemented tuff particulates in high nutrients medium.

Microalgae have shown a preference to attach and grow on Fe+Mn-supplemented tuff particulates over the control tuff particulates, due to the presence of the micronutrients on the tuff.

### Attachment and Growth of Microalgae on Fibrous Surfaces

Cotton fibers were used without further treatment (control) or pretreated by adsorption of Fe and Mn (Fe+Mn-supplemented cotton) Adsorbing Fe and Mn on cotton fibers was achieved by agitation of the fibers in a solution of Fe³⁺ (2 mM) and Mn²⁺ (0.2 mM) overnight and then washed in seawater 3 times before using for growth experiments. After washing, cotton fibers were incubated in low nutrient (LN) medium or high nutrient (HN) medium.

From day zero and until the 15th day of the experiment, a gradual growth was observed in all treatment groups, and the microalgae dry biomasses on Fe+Mn-supplemented cotton fibers higher than their respective controls. On the 15th day of the experiment, the microalgae grown on Fe+Mn-supplemented cotton fibers in the HN medium reached dry biomass of 8.26×10⁻³ gr biomass/gr particulate which was significantly higher compared to both the control group and the Fe+Mn-supplemented LN treatment group. Cotton supplemented with Fe³⁺ and Mn²⁺ (“Fe+Mn-supplemented”) in high nutrients medium exhibited a significantly higher biomass in comparison to control and in comparison to low nutrients medium.

On 15th day of the experiment, the microalgae dry biomass exhibited the most substantial differences between the treatment groups, with values 2.03-fold and 2.11-fold higher for Fe+Mn-supplemented cotton fibers in the HN medium compared to control cotton fibers in HN and LN media, respectively. Additionally, the biomass on Fe+Mn-supplemented cotton fibers in HN medium was 28.97-fold higher than that on control cotton fibers in LN medium.

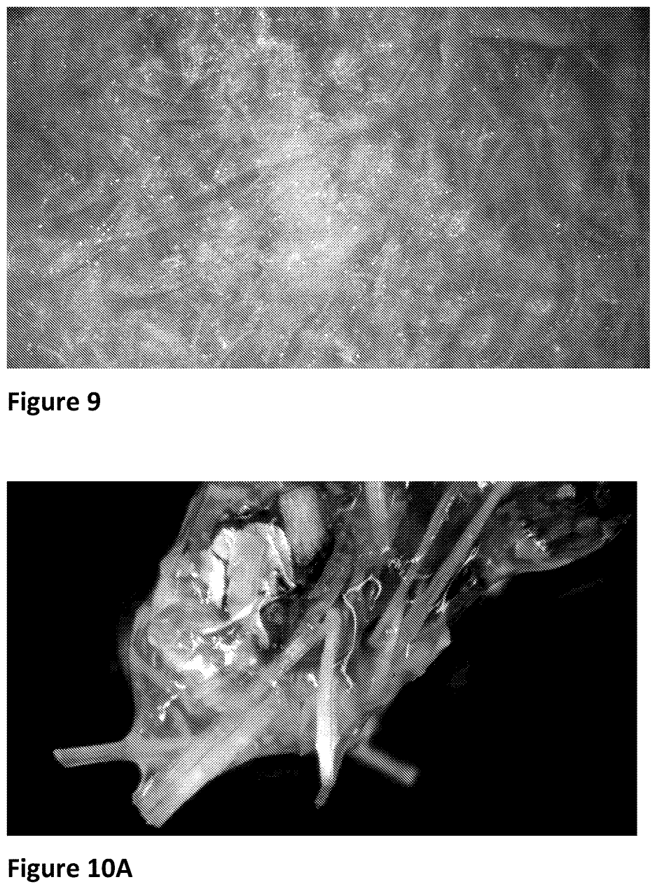

FIG. 9 shows microalgae (bright white spots) attached and entrapped within Fe+Mn-supplemented cotton fibers in HN medium (11th day) providing further evidence of high potential of cotton fibers for the attachment, entrapment, and growth of microalgae on and between its fibers.

### Example 3—Construction of the Multicomponent Particles

### Method:

Dry expanded vermiculite sieved to obtain particle sizes >500 μm was treated as follows: Vermiculite was placed in a 100 ml beaker, and 0.5M CaCl₂) solution in double deionized water (pH=3) was added. The mixture was filtered through a 500 μm sieve, and excess solution was removed by absorbing the water using a dry wipe.

A 2% Na alginate solution of ~150 ml was placed in a 250 ml beaker with a magnetic stirrer, stirred at 1000 rpm. Ca-washed vermiculite was gradually added to the alginate solution for 1 minute to obtain cross-linked, alginate-coated vermiculite. The alginate-coated vermiculite was then transferred to a Buchner funnel and washed thoroughly with double deionized water to remove excess alginate solution.

As a second stage, the scaffold was added. To this end, either cannabus or cotton fibers (hereinafter referred to as “fibers” for brevity) were cut into short threads (from hundreds of microns to few mm in length) and mixed with a 0.5% Na alginate solution in a beaker using a magnetic stirrer. Alternatively, particles were obtained with tuff dust of <500 μm fraction being mixed with a 0.5% Na alginate solution, without cotton fibers (i.e. tuff without fibers).

The coated vermiculite was briefly placed in a stirring CaCl₂) 0.5M solution (pH 3) and then filtered and gently dried to avoid damaging the coating and remove excess of CaCl₂) solution.

Subsequently, in order to adhere the alginate-coated vermiculite core with the fibers (either the cotton or the cannabus), the coated vermiculite particulates were transferred into a beaker containing fibers or tuff in Na alginate solution (the tuff dust in this example was attached to the coated vermiculite and not to the fibers). Finally, the fiber-coated or tuff-coated vermiculite particles were washed with double deionized water to remove excess alginate and expose the fibrous material or tuff surface.

### Results:

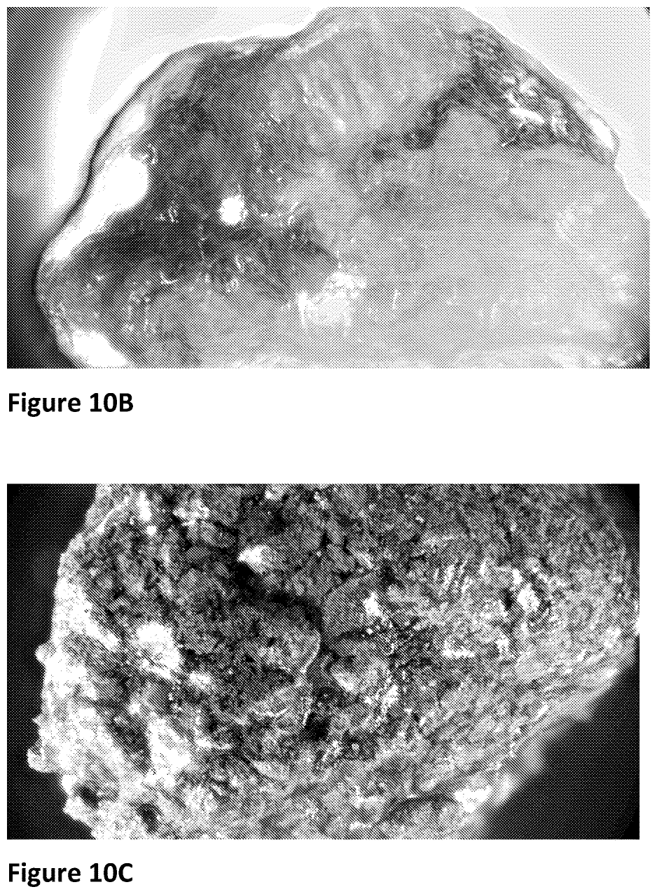

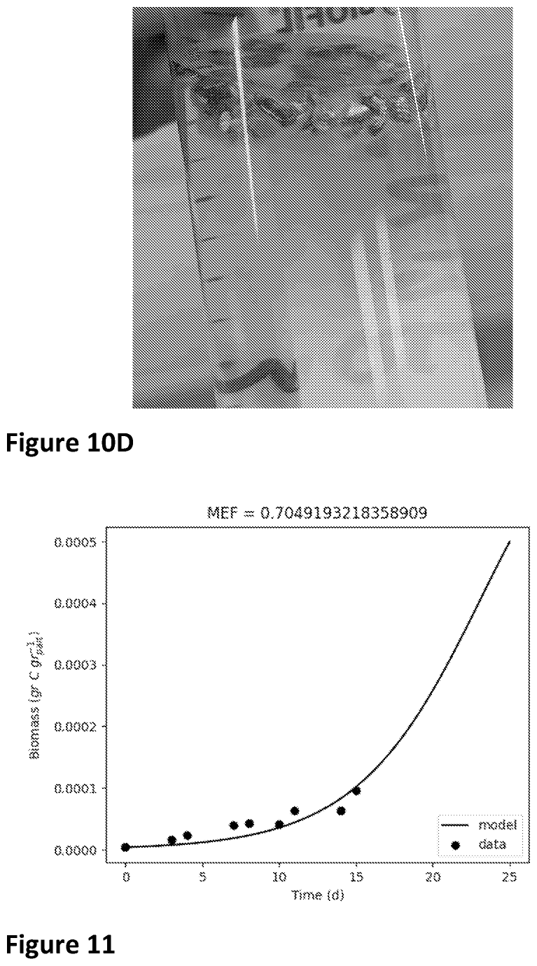

The fusion of fibrous materials with alginate-coated vermiculite resulted in the formation of stable particles with fibers comprising the external layer or with the tuff only, as shown in FIGS. 10A-10C. It was thus concluded that the fibers coated particles as well as the tuff coated particles can serve as a viable growth support in the presence of algae also when attached to the alginate barrier-coated vermiculate core.

It is noted that in the above examples, alginate was used also a binder for the scaffold over the already existing alginate barrier layer.

Subsequently, these cotton fiber/Ca-alginate/vermiculite particles were assessed for floatation/buoyancy. FIG. 10D demonstrated the ability to remain afloat for a minimum of 5 days, with the coating displaying considerable stability throughout this duration.

### Example 4—Iron Adsorption to Tuff Particles

The capacity of Tuff for iron adsorption and retention was assessed.

### Method:

To assess the iron adsorption capacity on tuff surface, tuff was incubated in a Fe³⁺ solution as described below. The amount of un-adsorbed Fe³⁺ that remained in solution after adsorption was then measured.

### The Adsorption Procedure

Tuff particulates (500-1000 μm fraction) were rigorously washed in double deionized water until achieving a clear supernatant. Then, 200 mL of freshly prepared Fe³⁺ solution (100 g L⁻¹) was added to the Tuff for an overnight incubation, after which the solution was decanted and the particulates were washed three times with DDW.

The tuff was then incubated in seawater for at least 30 minutes, followed by two additional seawater replacements, each with a minimum 30-minute incubation period. Dissolved iron measurements were taken in three stages: the initial 1 mM Fe³⁺ solution before tuff introduction, the solution after adsorption incubation, and seawater after each round of washing (designated as 1 for the first wash post-adsorption, and 2 and 3 for subsequent rounds). Dissolved iron was measured using Inductively Coupled Plasma Optical Emission Spectroscopy (ICP-OES) (Arcos FHM22 Spectro).

As control, tuff was treated in the same manner as samples without the addition of Fe³⁺ to the solution prior to incubation.

### Results:

Although tuff contains iron oxides in its structure, control tuff did not release dissolved iron to the solution as the oxide are insoluble.

Measurements of the iron concentration in the tested samples revealed that tuff effectively adsorbed the iron, leaving no detectable amount of dissolved iron in the solution following the incubation with tuff.

Furthermore, upon seawater washing, the control sample exhibited no release of iron into the solution, while the Fe-adsorbed tuff released minimal quantities of iron into the solution, indicating the nearly complete retention capability of tuff with respect to iron under the experimental concentrations employed.

### Example 5—Modelling Microalgae Growth on a Fiber

The growth of phytoplankton on a fibrous surface was described by the Fiber 2Particle model (as detailed below). The model is well-suited for comparison with surfaces of various designs.

The model consisted of a set of differential equations that represented biomass growth on the substrate, the availability of free macronutrients within the surrounding environment, and the uptake of micronutrients by the algae from the surface. The growth was constrained by the maximum attainable thickness and density of the biofilm that could be cultivated on the substrate surface.

To assess the sensitivity of the model to uncertain parameters, calibration was carried out using data obtained from laboratory experiments, focusing on the most sensitive parameters.

### Method:

Dataset: The dataset utilized in the model was derived from an experiment involving the growth and attachment of microalgae (Phaeodactylum tricornutum) onto vermiculite particles. An inoculum in the stationary phase was quantified using a hemocytometer and introduced into a HN medium (200 μM NaNO₃ and 20 μM KH₂PO₄) at the outset of the experiment. The experiment spanned a duration of 15 days and was conducted in duplicate.

Procedure: The experimental procedure encompassed replicates, each comprising a liquid phase (consisting of the diluted inoculum) and a solid phase (consisting of vermiculite particles). Vermiculite particulates were utilized in their untreated state as well as after undergoing pretreatment via Fe adsorption (100 gr of wet weight/liter). The initial algae cell concentration in the liquid phase was maintained at 2.5×10⁵ cells mL⁻¹.

These setups were placed in flasks positioned on an orbital shaker rotating at 75 RPM. Sampling was conducted four times per week. Microalgae present in the liquid phase were quantified using a hemocytometer. Solid-phase particles underwent rinsing in clean artificial seawater to remove unattached microalgae and were subsequently transferred to vials containing clean seawater. Following the washing procedure, the microalgae adhering to the particulates were extracted via 10 seconds of vortexing, followed by 12 seconds of sonication, and quantified using a hemocytometer.

Model description: The modeling of phytoplankton growth on a vermiculite particulate was conducted employing a fiber2particle model. Within this model, variables were incorporated to account for dissolved inorganic and organic carbon (C), nitrogen (N), and phosphorus (P) in the particle's environment. Furthermore, it considered the solid concentration of micronutrients such as iron (Fe) and manganese (Mn) on the particle, with the ecosystem being represented by phytoplankton. All parameter values were set to align with the growth conditions applied in the experiments, which featured P. tricornutum as the chosen algae.

The model accommodated variable stoichiometry in phytoplankton, specifically denoted as (C₁₀₆N₁₆P)₁₀₀₀Fe₈Mn₁₄, commonly known as the Redfield ratio. This OD (zero-dimensional) offline model, was set and executed over a period of 25 days, assuming a particle density of N=100 particles per liter of seawater. The model was implemented using Python (version 3.10).

The Monod (1949) phytoplankton growth rate function was utilized to depict the growth of phytoplankton on a particle, as articulated in Equation 1:

$$μ = {μ}^{max} \cdot {\prod }_{i = C , N , P , I} \frac{[ i ]}{[ i ] + {K}_{i}} \cdot {\prod }_{j = Fe , Mn} \frac{[ i ]}{[ i ] + {K}_{j}^{′}} \qquad ( 1 )$$

where μ is the specific growth rate (d⁻¹), and μ^{max} is the maximum growth rate assuming no limiting factors (d⁻¹). μₘₐₓ is then multiplied by the Monod expressions of the different elemental limiting factors (i=C, N, P) and the light limitation (l) where [i] represents the element concentration (μmol L⁻¹) or light intensity (μE), respectively, and Kᵢ is the Monod constant [Monod, J. 1949. Growth of bacterial cultures. Annu. Rev. Microbiol. 3:371-394. doi: 10.1146/annurev.mi.03.100149.002103]. Although iron (Fe) and manganese (Mn) are not considered to be limiting factors on the particle surface, they were included in the equation in a Monod style fashion that includes the parameter K′j, is where the concentration of j=Fe, Mn that cannot support growth.

The basic biomass growth is described by Equation 2:

$$\frac{dB}{dt} = μB - respB \qquad ( 2 )$$

where B is the phytoplankton carbonic biomass (gr C grₚₐᵣₜ⁻¹), and resp is the phytoplankton respiration rate (d⁻¹). In addition, to the biomass growth, differential equations were used to describe the changes in concentrations per day for the macronutrients in the environment (N, P, C) and the micronutrients on the particles (Fe and Mn).

The growth around the fiber was restricted by the maximum thickness that is observed in nature for biofilm, R=100 μm and the integration of the differential equations was then terminated when reached 10%, as biofilm in nature consists of about 10% dry mass.

Sensitivity analysis and calibration: Some parameters had a large uncertainty to their literature values, and therefore were detected first with a linear sensitivity analysis (LSA). To systematically test to what extent these specific parameters affect the model's output, each parameter was given a set of 10 random values from a literature range.

The model was run for each value and parameters that showed great variation in model's output, were chosen for calibration.

The calibration was performed with Latin Hypercube sampling (LHS), a statistical method for generating a near-random sample of parameter values from a multidimensional distribution. We established 1000 combination sets of the chosen parameters. We used the same parameter ranges as we used in the LSA. Each set was evaluated with a simple sum-of-squares (SS) serving as cost-function, and the set that yielded the minimum SS value was chosen as the set for calibration.

To better evaluate the best fit, we calculated the model efficiency (MEF):

$$MEF = 1 - \frac{{\sum }_{i = 1}^{N} {( {P}_{i} - {O}_{i} )}^{2}}{{\sum }_{i = 1}^{N} {( {O}_{i} - \overline{O} )}^{2}} ,$$

where: Pᵢ and Oᵢ denote the predicted value and observed value i, and O is the observed value mean. Values of MEF close to 1 represent a very good fit, values close to 0 mean that the prediction is no better than the mean of the observations, and values smaller than 0 mean that the prediction is worse than the mean of the observations.

### Results:

The model results of the parameter set that yielded the minimum SS value after n=1000 samples of Latin Hypercube Sampling (LHS) sets are presented in FIG. 11. Specifically, FIG. 11 depicts the model results illustrating algae biomass growth on fibrous particles. The solid line corresponds to the model outcomes, while the dots represent data acquired from laboratory experiments. The best fit showed model efficiency value of MEF=0.705 to the cell number measured in the laboratory experiment.

The strong alignment with the laboratory results indicates the utility of the model for predicting growth in diverse setups and conditions.

1. A population of particles, each particle comprising
a construct comprising:
at least one solid core,
optionally, at least one barrier coating layer over the at least one solid core, and
a scaffold associated at least at the outer surface of said at least one solid core or of said barrier coating, if said barrier coating is present in the construct, the scaffold being suitable for support growth of photosynthesizing aquatic organism;
gas entrapped within said at least one solid core, the gas having a first specific gravity less than that of water and present in an amount sufficient to provide floatation of said particle, once the particle is brought into contact with the water;
said construct having a second specific gravity greater than that of water; and
said at least one solid core, or said barrier coating, if present in said construct, having a permeability configured to allow controlled exchange between the entrapped gas and water external to said construct.

2. The population of particles of claim 1, wherein said solid core fulfills any one of: (i) it comprises or is expanded or porous particulate; (ii) it comprises or is a particulate organic core; or (iii) it comprises or is a particulate hydrocolloid.

3.-9. (canceled)

10. The population of particles of claim 2, wherein said particulate organic core is an organic core selected from the group consisting of carbon-based sponge, carbon-based foam, carbon-based fibrous material.

11.-19. (canceled)

20. The population of particles of claim 1, comprising at least one layer of said barrier coating over the at least one solid core.

21. The population of particles of claim 20, wherein the at least one layer of barrier coating: (i) comprises or is a hydrocolloid coating or (ii) comprises a long chain organic substance.

22-33. (canceled)

34. The population of particles of claim 1, wherein said scaffold comprises a nutrient composition suitable for supporting growth of photosynthesizing aquatic organism.

35. The population of particles of claim 34, wherein said nutrient composition comprises at least one nutrient selected from the group consisting of iron (Fe), Zinc (Zn), Copper (Cu), Manganese (Mn), Molybdenum (Mo), Selenium (Se), Chromium (Cr), Cobalt (Co), Iodine (I), Fluorine (F), Magnesium (Mg), Silicon (Si), Nitrogen (N), Phosphorus (P), Sulfur (S), Strontium (Sr), Nikel (Ni), Vanadium (V) and any combination of same.

36.-37. (canceled)

38. The population of particles of claim 1, wherein said scaffold comprises any one or combination of fibers and water-insoluble porous particulate material.

39. The population of particles of claim 1, wherein said fibers are organic fibers.

40-59. (canceled)

60. The population of particles of claim 1, wherein said barrier layer is a binder layer or said particles comprises a binder over and/or within said at least one layer of barrier coating and said binder binds said scaffold to the solid core.

61.-62. (canceled)

63. The population of particles of claim 1, wherein said gas is selected from the group consisting of air and CO₂.

64. The population of particles of claim 1, wherein said exchange between the entrapped gas and water external to said construct is controlled by at least one parameter selected from the group consisting of material of core, dimension of core, surface area of core, porosity of core, specific gravity of core, surface energy of core, wettability of core, number of layers of said barrier coating; gas permeability of said at least one layer of barrier coating, water permeability of said at least one layer barrier coating, solubility of said at least one layer of barrier coating, thickness of said at least one layer of barrier coating, overall thickness of said barrier layer, composition of said at least one layer of barrier coating; wettability of said at least one layer of the barrier coating, overall wettability of the barrier coating, type of entrapped gas, amount of entrapped gas, water permeability of the barrier coating, water resistance of the barrier coating, gas permeability of the barrier coating, gas resistance of the barrier coating, outer surface charge, outer surface polarity, outer surface free energy and any combination of same.

65.-68. (canceled)

69. A method of producing a population of particles, the method comprising mixing solid core material, optionally having at least one layer of barrier coating over the solid core, with a scaffold forming material under conditions suitable to allow association of the scaffold material to at least an outer surface of the solid core;
wherein said solid core material comprises gas entrapped therein, the gas having a first specific gravity less than that of water and is in an amount sufficient to result in floatation of said particle, once the particle is brought into contact with the water;
wherein the combination of at least one solid core, said at least one layer of barrier coating, if present, and the scaffold having a second specific gravity greater than that of water; and
wherein the combination of the solid core, the at least one layer of barrier coating, if present, the scaffold and the gas is selected to provide, in the resulting particle, controlled exchange between the entrapped gas and water external to said construct, once said particle is brought into contact with water.

70-71. (canceled)

72. The method of claim 69, wherein said solid core fulfills any one of: (i) it comprises or is expanded or porous particulate (ii) it comprises or is a particulate organic core, or (iii) it comprises or is a particulate hydrocolloid.

73.-86. (canceled)

87. The method of claim 69, comprising supplementing at least said scaffold with a nutrient composition.

88.-90. (canceled)

91. The method of claim 69, comprising fixedly attaching water insoluble porous material to said fibers and/or to said barrier coating.

92.-106. (canceled)

107. The method of claim 69, comprising applying a binder within and/or over said at least one layer of barrier coating, if said at least one layer of barrier coating is present, or over said solid core, prior to or concomitant with association of the scaffold.

108.-112. (canceled)

113. The method of claim 69, comprising active introduction of said gas into said solid core.

114.-119. (canceled)

120. The method of claim 69, comprising selecting particles having a size below 1 cm.

121. A method for carbon dioxide sequestration, comprising distributing a population of particles over a selected area of body of water comprising at least one photosynthesizing aquatic organism and being open to a source of carbon dioxide to be sequestered; wherein said population of particles comprise a construct comprising:
at least one solid core,
optionally, at least one barrier coating layer over the at least one solid core, and a scaffold associated at least at the outer surface of said at least one solid core or of said barrier coating, if said barrier coating is present in the construct, the scaffold being suitable for support growth of the photosynthesizing aquatic organism;
gas entrapped within said at least one solid core, the gas having a first specific gravity less than that of water and present in an amount sufficient to provide floatation of said particle, once the particle is brought into contact with the water;
said construct having a second specific gravity greater than that of water; and
said at least one solid core, or said barrier coating, if present in said construct, having a permeability configured to allow controlled exchange between the entrapped gas and water external to said construct.

122.-124. (canceled)
